SEQUENCE LISTING (3 sequences)

SEQ ID NO: 1 - RNA, 50 nt, synthetic construct
  source 1..50: mol_type = other RNA
gggctaagcgataaaccgaagctgcgggtgtgtactatgtacacgcggta

SEQ ID NO: 2 - RNA, 50 nt, synthetic construct
  source 1..50: mol_type = other RNA
gccaccccaaaaatgaaggggactaaaacacttctaaccgaggtcgaaac

SEQ ID NO: 3 - RNA, 50 nt, synthetic construct
  source 1..50: mol_type = other RNA
gccaccccaaaaatgaaggggactaaaacatgtccgggccgggtgaggtt

(19) United States
(12) Patent Application Publication
(10) Pub. No.: US 2025/0043332 A1
(43) Pub. Date: Feb. 6, 2025

(54) METHODS FOR DETECTING NUCLEIC ACID TARGETS

(71) Applicant: The Charles Stark Draper Laboratory, Inc., Cambridge, MA (US)

(72) Inventors: Isaac W. Moran, Maynard, MA (US); Michael Turo, Cambridge, MA (US)

(21) Appl. No.: 18/778,744

(22) Filed: Jul. 19, 2024

Related U.S. Application Data
(60) Provisional application No. 63/517,308, filed on Aug. 2, 2023.

(51) Int. Cl. C12Q 1/6816

(52) U.S. Cl. CPC C12Q 1/6816

(57) ABSTRACT

Methods, microarrays, and kits for detecting target nucleic acid molecules (e.g., in a sample) using immobilized guide RNA (gRNA), or gRNA/Cas complexes, are provided herein. In some embodiments disclosed herein, a plurality of guide RNA (gRNA) molecules are bound, directly or indirectly, to a solid support, wherein at least one gRNA molecule in the plurality comprises a nucleotide sequence that is complementary to a portion of the target nucleic acid in the sample.

### RELATED APPLICATION(S)

This application claims the benefit of U.S. Provisional Application No. 63/517,308, filed on Aug. 2, 2023. The entire teachings of the above application(s) are incorporated herein by reference.

### GOVERNMENT SUPPORT

This invention was made with Government support under Contract No. N66001-21-C-4048 awarded by the US Defense Advanced Research Projects Agency (DARPA). The Government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 50001071-001_SL.xml; created Jul. 19, 2024, 3,583 Bytes in size.

### TECHNICAL FIELD

Methods for detecting target nucleic acid molecules (e.g., covalently labeled target nucleic acid molecules) using guide RNAs or guide RNA (gRNA)/Cas complexes (a guide RNA molecule bound to a Cas enzyme) on a solid support are provided herein. Microarrays and kits for carrying out the disclosed methods are also provided herein.

### BACKGROUND

There is a need for assays to detect nucleic acids that are present at low concentrations in samples. Sensitive nucleic acid detection methods can assist in early and accurate detection of pathogens and/or diagnosis of diseases, enhancing the effectiveness of medical interventions and treatments. Commonly used techniques for nucleic acid detection include quantitative polymerase chain reaction due to its high sensitivity and specificity in detecting low quantities of DNA. Improving the sensitivity and reliability of DNA detection methods is a continued area of research.

### SUMMARY

As described herein, guide RNA (gRNA) and gRNA/Cas complexes are useful for detecting target nucleic acids in a sample, even at very low concentrations. The cleavage activity of Cas enzymes can be deactivated in several ways such that the target nucleic acid is not cleaved upon binding. Cas enzymes can aid in the capture efficiency of target nucleic acids, such as pathogen DNA or RNA, at low concentrations. An improved assay that is capable of detecting multiple targets at once, i.e., a multiplex assay, is desirable.

Disclosed herein are methods of detecting a target nucleic acid in a sample. In some embodiments, the methods comprise: providing a sample comprising a target nucleic acid; contacting the sample with an apparatus comprising a plurality of guide RNA (gRNA) molecules bound, directly or indirectly, to a solid support, wherein at least one gRNA molecule in the plurality comprises a nucleotide sequence that is complementary to a portion of the target nucleic acid in the sample, and wherein the contacting is under conditions in which the target nucleic acid hybridizes to the complementary gRNA molecule on the apparatus; washing the apparatus to remove unhybridized nucleic acids; and detecting the target nucleic acid.

In some embodiments, the methods of detecting a target nucleic acid in a sample comprise: providing a sample comprising a target nucleic acid, wherein the target nucleic acid comprises a label and is present in the sample at a concentration of about 1 nM or less; contacting the sample with a microarray comprising a plurality of guide RNA (gRNA) molecules covalently bound to a solid support, for a time period of about 24 hours or less, wherein: the plurality of gRNA molecules are in a complex with a deactivated CRISPR-associated (Cas) enzyme, at least one gRNA molecule in the plurality comprises a nucleotide sequence that is complementary to a portion of the target nucleic acid in the sample, and the contacting is under conditions in which the target nucleic acid hybridizes to the complementary gRNA molecule on the apparatus; washing the apparatus to remove unhybridized nucleic acids; and detecting the target nucleic acid using the label.

Also disclosed herein are microarrays comprising a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid.

Further disclosed herein are kits comprising: a microarray comprising a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid.

### BRIEF DESCRIPTION OF THE DRAWINGS

The foregoing will be apparent from the following more particular description of example embodiments, including those illustrated in the drawings interspersed herein. The drawings are not necessarily to scale, emphasis instead being placed upon illustrating embodiments.

FIG. 1 provides a scheme illustrating an example embodiment of a method disclosed herein of RNA target binding and detection by an immobilized dRNA/Cas complex.

FIG. 2 shows slide microarray layouts for an example d-gRNA/Cas13 assay for detecting labeled RNA.

FIG. 3 provides a scheme depicting an example workflow for a d-gRNA/Cas based assay.

FIG. 4 shows plots of concentration series for three different targets tested on micro-arrays on which an example embodiment of a method disclosed herein was performed. Three replicates are depicted from each target with 18s (a human biomarker), FluA, and BH (Bordetella holmesii).

FIG. 5 shows multiplexing plots for an example d-gRNA/Cas/Target-AF488 Assay on which an example embodiment of a method disclosed herein was performed of 6×10⁸ copies/μL for BH (Bordetella holmesii), 18s (a human biomarker) and FluA (a viral pathogen).

FIG. 6 shows representative fluorescent images of multiplexed assays for duplexed (a-c), triplexed (d), and (e) off-target binding on which an example embodiment of a method disclosed herein was performed.

FIG. 7 shows plots for off-target interactions with an example d-gRNA/Cas/target-AF488 assay utilizing R440 (a sequence from Chlamydia) as target at a concentration of 6×10⁸ copies/μL on which an example embodiment of a method disclosed herein was performed. Three replicates are depicted from each target. The three targets were 18s, FluA, and BH (Bordetella holmesii).

FIG. 8 provides a schematic illustrating an example embodiment of a method disclosed herein of quantum dot target labeling after capture by glass substrate supported Cas complex. FIG. 8 illustrates primary amine d-gRNA oligos anchored to epoxide functional glass via amine/epoxy coupling and ensuing complex formation with Cas enzyme. The immobilized d-gRNA/Cas complex is utilized to capture biotin labeled targets which are then labeled with streptavidin conjugated quantum dots.

FIG. 9 provides representative fluorescent images of initial attempts at labeling captured synthetic BH (Bordetella holmesii) target with quantum dots on a microarray on which an example embodiment of a method disclosed herein was performed. On left, unblocked quantum dot labeling of d-gRNA (BH)/Cas subarrays after target capture for subarray with varying amounts of d-gRNA (BH). Without blocking, significant non-specific binding from the quantum dots was observed. On right, blocking with BSA was shown to reduce quantum dot non-specific binding and enable target specific signal from quantum dot labeling of on-target subarray for the BH-biotin target.

FIG. 10 provides a representative fluorescent image of captured synthetic BH (Bordetella holmesii) target labeled with quantum dots on a microarray on which an example embodiment of a method disclosed herein was performed. The image shows on-target emission for synthetic BH-biotin captured by d-gRNA (BH)/Cas subarrays. Blocking with SuperBlock™ or Pierce™ Non-Protein Blocker improved the signal over blocking with BSA, with Non-Protein blocker yielding the lowest background.

FIGS. 11A-11B show quantification of signal from FIG. 10 and degree of signal amplification from quantum dots over molecular AF488.

FIGS. 12A-12B show quantification of signal for the detection of genomic BH (Bordetella holmesii)-biotin with quantum dot labeling from a microarray on which an example embodiment of a method disclosed herein was performed. Plots for 2 attempts at the detection of genomic BH-biotin are depicted with inclusion of gelatin as a screening agent against non-specific target interactions. Each plot contains background subtracted emission for on-target d-gRNA (BH) subarrays as well as off-target (d-gRNA (Flu), d-gRNA (18s)) subarrays.

FIG. 13 shows a comparison of fluorescent signal for microarrays with and without Cas enzyme for various genomic targets (BH, Flu, and 18s) at various concentrations.

FIG. 14 shows quantification of signal (after subtracting background signal) for the detection of various genomic targets (BH, Flu, and 18s) with and without Cas enzyme at various target concentrations.

### DETAILED DESCRIPTION

A description of example embodiments follows.

### I. Definitions

The meaning of some terms and phrases used in the specification, examples, and claims are provided below. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

It is understood that the specific order or hierarchy of steps in the methods or processes disclosed is an illustration of example approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.” The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element or step not specified in the claim; and (iii) “consisting essentially of” limits the scope of a claim to the specified materials or steps “and those that do not materially affect the basic and novel characteristic(s)” of the claimed invention and disclosure. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the disclosure, claims, result or embodiment, “about” means within one standard deviation per the practice in the art, or can mean a range of ±20%, ±10%, ±5%, ±4, ±3, ±2 or ±1% of a given value. It is to be understood that the term “about” can precede any particular value specified herein, except for particular values used in the Examples.

As used herein, “nucleic acid” means DNA, RNA, single-stranded, double-stranded, or more highly aggregated hybridization motifs, and any chemical modifications thereof.

As used herein, “synthetic nucleic acids,” “synthetic DNA” or “synthetic RNA,” refers to lab-created nucleic acids. Synthetic nucleic acids are frequently shorter than genomic nucleic acids.

As used herein “genomic nucleic acids,” “genomic DNA,” or “genomic RNA” as used herein, refers to nucleic acids extracted from a natural source, such as from cultured bacteria. Genomic nucleic acids can widely vary in length and are frequently longer than synthetic nucleic acids.

As used herein, a “target nucleic acid” or “target RNA” or “target DNA” refers to a nucleic acid in a target gene that has complementarity to the guide sequence of the gRNA. The interaction of the target nucleic acid sequence and the guide sequence directs an RNA-guided DNA binding agent to bind, and potentially nick or cleave (depending on the activity of the agent), within the target sequence.

“Guide RNA”, “gRNA”, and simply “guide” are used herein interchangeably to refer to the guide that directs an RNA-guided DNA binding agent to a target DNA and can be cither a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally occurring sequence, or a trRNA sequence with modifications or variations compared to naturally occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region contain one mismatch. In some embodiments, the guide sequence and the target region may contain 1-4 mismatches where the guide sequence comprises at least 17, 18, 19, 20 or more nucleotides. In some embodiments, the guide sequence and the target region may contain 1 mismatch where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region contain 1 mismatch where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the protospacer adjacent motif (PAM) except for the substitution of U for T in the guide sequence.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Example alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

As used herein, “Cas enzyme” or “Cas enzymes” refers to a CRISPR-associated (Cas) endonuclease, i.e., an enzyme, that acts to cut or nick or cleave DNA or RNA at a location specified by a guide RNA.

As used herein, “deactivated Cas enzyme” or “deactivated Cas enzymes” or “dead Cas” or “dCas” or “d-Cas” refers to a Cas enzyme that no longer cuts or nicks or cleaves DNA or RNA, or has significantly reduced ability to cut or nick or cleave DNA or RNA. Typically, binding of the deactivated Cas enzyme is not impacted by deactivation.

As used herein, “deactivating guide RNA” or “d-gRNA” refers to a guide RNA sequence is complementary to a target nucleic acid, but not sufficiently complementary to allow Cas to start cleavage, cutting, or nicking of the target nucleic acid. In other words, the d-gRNA deactivates the Cas enzyme's cutting or nicking or cleaving ability. Deactivating guide RNAs are described in Tambe, A. et al., “RNA Binding and HEPN-Nuclease Activation Are Decoupled in CRISPR-Cas13a,” Cell Reports 24:1025-1036 (2018), which is incorporated herein by reference for its description of deactivating guide RNAs.

As used herein, a “label” refers to a moiety or compound joined directly or indirectly to a nucleic acid, such as a target nucleic acid, that is detected or leads to a detectable signal. Direct labeling can occur through bonds or interactions that link the label to the nucleic acid, including covalent bonds or non-covalent interactions, e.g., hydrogen bonds, hydrophobic and ionic interactions, or formation of chelates or coordination complexes. Indirect labeling can occur through use of a bridging moiety or “linker” such as a binding pair member, an antibody or additional nucleic acid, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), or fluorophore. More than one label, and more than one type of label, may be present on a particular nucleic acid, or detection may use a mixture of oligomers in which each oligomer is labelled with a compound that produces a different detectable signal.

As used herein, “Mirus® reagent” refers to a chemical structure comprising: (1) a label; (2) a linker that facilitates electrostatic interactions with nucleic acids; and (3) a reactive alkylating group that covalently attaches to any reactive heteratom with the nucleic acid.

As used herein, a “Mirus® linker” refers to a linker that does not appreciably disrupt electrostatic interactions between hybridizing chains of nucleic acids, and has a reactive alkylating group that covalently attaches to any reactive heteratom with the nucleic acid. A “Mirus® linker” attaches a label to a nucleic acid, but the term “Mirus® linker” as used herein does not include a label.

As used herein, “microarray” refers to a solid support (e.g., a glass or epoxy slide) with the controls and/or gRNAs printed or dispensed on it.

“Sample” refers to any material that may contain or is suspected of containing one or more target biological material, such as bacterial or viral components, such as nucleic acids or fragments of nucleic acids. A sample may be a complex mixture of components. Samples include “biological samples” which include any tissue or material derived from a living or dead mammal or organism, including, for example, stool, blood, plasma, serum, blood cells, saliva, mucous and cerebrospinal fluid. Samples may also include samples of in vitro cell culture constituents including, for example, conditioned media resulting from the growth of cells and tissues in culture medium. Samples also include food or beverage, including water and other aqueous solutions, which includes any material intended or suitable for consumption. Samples also include material taken from the environment, such as water. In one step of the methods described herein, a sample is provided that is suspected of containing at least one target biological material, such as bacterial or viral target nucleic acid. Accordingly, this step excludes the physical step of obtaining the sample from a subject.

As used herein, “blocking solution” refers to a solution for preventing non-specific protein binding to a surface.

Although methods and materials similar or equivalent to those provided herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Where a range of values is provided, each numerical value between the upper and lower limits of the range is contemplated and disclosed herein.

Although certain embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The description of embodiments of the disclosure is not intended to be exhaustive or to limit the disclosure to the precise form disclosed. While specific embodiments of, and examples for, the disclosure are disclosed herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments disclosed herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. It will be apparent to those skilled in the relevant art that various modifications, additions, substitutions, and the like can be performed without altering the spirit or scope of the invention, and such modifications and variations are encompassed within the scope of the invention as defined in the claims which follow.

### II. Methods of Detecting Nucleic Acids

The present disclosure generally relates to methods of detecting a target nucleic acid in a sample (e.g., a biological sample), comprising one or more of: providing a sample comprising a target nucleic acid (e.g., DNA, RNA); contacting the sample with an apparatus (e.g., a microarray comprising multiple identifiable locations) comprising a plurality of nucleic acid (e.g., guide RNA (gRNA)) molecules bound, directly or indirectly, to a solid support, wherein at least one nucleic acid (e.g., gRNA) molecule in the plurality comprises a nucleotide sequence that is complementary to a portion of the target nucleic acid in the sample, and wherein the contacting is under conditions in which the target nucleic acid hybridizes to the complementary nucleic acid (e.g., gRNA) molecule on the apparatus; washing the apparatus to remove unhybridized nucleic acids; and detecting the target nucleic acid. In some embodiments, the apparatus is a microarray comprising multiple identifiable locations.

In some embodiments, the at least one nucleic acid (e.g., gRNA) molecule in the plurality of nucleic acid molecules that is bound, directly or indirectly, to a solid support comprises a nucleotide sequence that is complementary to about 1% to about 100% (e.g., about 10% to about 100%, about 20% to about 100%, about 30% to about 100%, about 50% to about 100%, about 60% to about 100%, about 70% to about 100%, about 80% to about 100%, etc.) of the target nucleic acid in the sample.

In some embodiments, the sample comprises a plurality of different target nucleic acids and the method comprises detecting one or more of the different target nucleic acids. In some embodiments, the apparatus is a microarray comprising gRNA molecules having nucleic acid sequences that are complementary to each of the plurality of different target nucleic acids in the sample. In some embodiments, the nucleic acid sequences are complementary to about 1% to about 100% (e.g., about 10% to about 100%, about 20% to about 100%, about 30% to about 100%, about 50% to about 100%, about 60% to about 100%, about 70% to about 100%, about 80% to about 100%, etc.) of each of the plurality of different target nucleic acids in the sample.

In some embodiments, the sample is a biological sample. In some embodiments, the biological sample comprises a pathogen or nucleic acid from a pathogen. In some embodiments, the biological sample is a clinical sample from a subject (e.g., a human) or group of subjects.

In some embodiments, the target nucleic acid is DNA. In other embodiments, the target nucleic acid is RNA. In some embodiments, the target nucleic acid comprises a label (e.g. a quantum dot, a fluorescent molecule, biotin). The label may be conjugated to the target nucleic acid using a linker (e.g., Mirus® linker, a linker comprising quaternary amine). In some embodiments, the label comprises a quantum dot. In some embodiments, the label comprises a fluorescent molecule. In some embodiments, the label comprises biotin. In some embodiments, the label is conjugated to the target nucleic acid using a linker.

The target nucleic acid may also be unlabeled. Methods of the present disclosure further comprise incubating a target nucleic acid (e.g., an unlabeled target nucleic acid) with a labeled oligonucleotide. In some embodiments, the labeled oligonucleotide comprises a nucleotide sequence that is complementary to a portion of the target nucleic acid.

In some embodiments, methods of the present disclosure further comprise incubating an unlabeled target nucleic acid with a labeled oligonucleotide, wherein the labeled oligonucleotide comprises a nucleotide sequence that is complementary to a portion of the target nucleic acid.

In some embodiments, the labeled oligonucleotide comprises a nucleotide sequence that is complementary to about 1% to about 100% (e.g., about 10% to about 100%, about 20% to about 100%, about 30% to about 100%, about 50% to about 100%, about 60% to about 100%, about 70% to about 100%, about 80% to about 100%, etc.) of the target nucleic acid.

In some embodiments, the target nucleic acid is present in the sample at a concentration of about 100 mM or less (e.g., about 10 mM or less, about 1 mM or less, about 100 nM or less, about 10 nM or less, about 1 nM or less, about 100 pM or less, about 1 pM or less, etc.). In some embodiments, the target nucleic acid is present in the sample at a concentration of about 1 pM to about 100 mM (e.g., about 1 pM to about 50 mM, about 1 pM to about 10 mM, about 1 pM to about 1 mM, about 1 pM to about 100 nM, about 1 pM to about 10 nM, about 10 pM to about 1 nM, about 1 pM to about 1 nM, about 1 pM to about 100 pM, about 1 pM to about 10 pM, etc.). In some embodiments, the target nucleic acid is present in the sample at a concentration of about 100 pM to about 1 nM. In other embodiments, the target nucleic acid is present in the sample at a concentration of about 1 pM to about 100 pM. In some embodiments, the target nucleic acid is present in the sample at a concentration of about 1 nM or less. In some embodiments, the target nucleic acid is present in the sample at a concentration of about 100 pM or less.

In some embodiments, the sample is contacted with the apparatus for a time period of about 72 hours or less (e.g., 48 hours or less, 24 hours or less, 10 hours or less, 5 hours or less, 1 hour or less). In some embodiments, the sample is contacted with the apparatus for a time period of about 1 minute to about 72 hours (e.g., about 1 minute to about 48 hours, about 1 minute to about 36 hours, about 1 minute to about 24 hours, about 1 minute to about 12 hours, about 1 minute to about 5 hours, about 1 minute to about 3 hours, about 1 minute to about 1 hour, about 1 minute to about 45 minutes, about 1 minute to about 30 minutes, etc.). In some embodiments, the sample is contacted with the apparatus for a time period of about 24 hours or less. In some embodiments, the sample is contacted with the apparatus for a time period of about 1 hour or less. In other embodiments, the sample is contacted with the apparatus for a time period of about 1 minute to about 1 hour.

In some embodiments, a plurality of nucleic acid (e.g., gRNA) molecules on the apparatus are modified. The 5′ and/or 3′ end(s) of the nucleic acid molecules may be modified with various chemical groups (e.g., amine, thiol, aldehyde, etc.). Various strategies of immobilizing nucleic acid molecules on the apparatus are also contemplated herein, for example, adsorption methods, covalent bonding, non-covalent strategies such as avidin/streptavidin-biotin interactions, etc.). In some embodiments, the plurality of gRNA molecules on the apparatus are modified. In some embodiments, the plurality of gRNA molecules on the apparatus are 5′ amine-modified.

In some embodiments, the plurality of gRNA molecules on the apparatus are in a complex with a CRISPR-associated (Cas) enzyme. Non-limiting examples of Cas enzymes include Cas1, Cas IB. Cas2, Cas3, Cas4, Cas5, Cas5e (CasD), Cas6, Cas6e, Cas6f, Cas7, Cas8, Cas8a1, Cas8a2, Cas8b, Cas8c, Cas9 (also known as Csn1 and Csx12), Cas10, Cas10d, Cas13, Cas13a, Cas13c, CasF, CasH, Csy1, Csy2, Csy3, Cse1, Cse2, Cse3, Cse4, Cse1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Csz1, Csx3, Csx1, Csx15, Csf1, Csf2, Csf3, Csf4, Cul966, Cpf1, C2c1, C2c3, homologs thereof, modified versions thereof, and combinations thereof. In some embodiments, the Cas enzyme is a class 2 Cas enzyme (e.g., type II, V, VI). In some embodiments, the Cas enzyme is a Cas9, Cas12, or Cas13 enzyme. The enzyme may be a Cas (e.g., Cas9, Cas12, Cas13, etc.) homolog or ortholog.

In some embodiments, the Cas enzyme is a deactivated Cas enzyme. In some embodiments, the deactivated Cas enzyme does not possess endonuclease activity but retains the ability to bind the target nucleic acid in a site-specific manner and can be generated, for example, by introducing point mutations in catalytic residues (e.g., in D10A and H840A. of the gene encoding Cas9). In some embodiments, the deactivated Cas enzyme is a deactivated Cas9 enzyme, a deactivated Cas12 enzyme, or a deactivated Cas13 enzyme. In some embodiments, the deactivated Cas enzyme is a deactivated Cas13 enzyme.

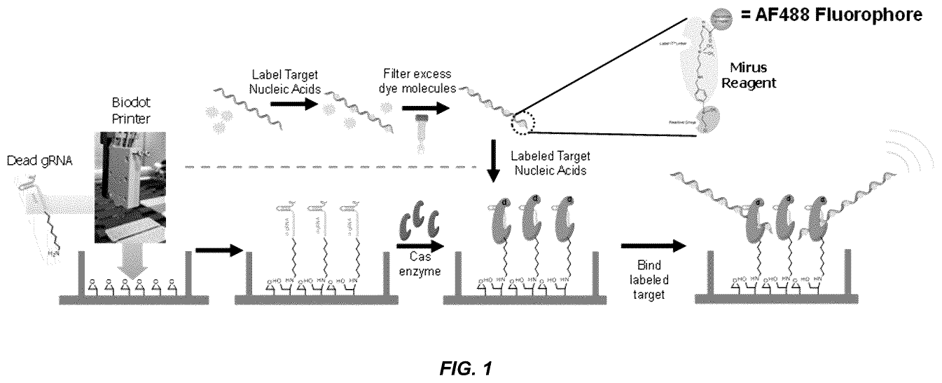

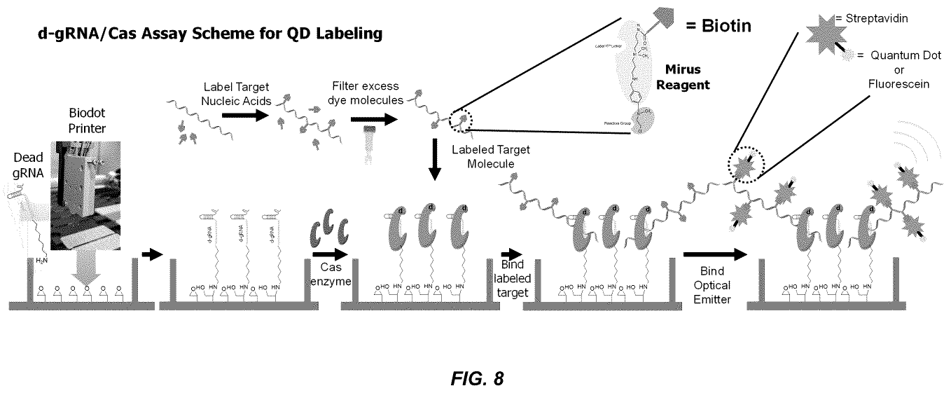

In some embodiments, a method of detecting a target nucleic acid in a sample comprises first labeling a target nucleic acid with a label. Second, after labeling the target nucleic acid, applying the labeled target nucleic acid to a microarray. Third, on the microarray, incubating the target nucleic acid with a guide RNA (gRNA) and a deactivated enzyme, wherein the gRNA is bound, directly or indirectly, to a solid support, wherein at least a portion of the gRNA is complementary to the target nucleic acid, and further wherein the gRNA and the Cas enzyme form a complex. The method also comprises washing the microarray and detecting the target nucleic acid. FIG. 1 and FIG. 8, for example, provide illustrations of embodiments of methods disclosed herein.

In some embodiments, a method of detecting a target nucleic acid in a sample comprises applying target nucleic acids to a microarray; then labeling the target nucleic acids; and on the microarray, incubating the target nucleic acid with a guide RNA (gRNA) and a deactivated Cas enzyme, wherein the gRNA is bound, directly or indirectly, to a solid support, wherein at least a portion of the gRNA is complementary to the target nucleic acid, and further wherein the gRNA and the Cas enzyme form a complex.

In some embodiments, a method of detecting a target nucleic acid in a sample comprises incubating target nucleic acids with a guide RNA (gRNA), wherein at least a portion of the gRNA is complementary to the target nucleic acid, and labeling the target nucleic acids; then applying target nucleic acids and gRNA to a solid support to bind directly or indirectly to the solid support; and then applying a deactivated Cas enzyme, wherein the gRNA and the Cas enzyme form a complex.

In some embodiments, a method of detecting a target nucleic acid in a sample comprises incubating target nucleic acids with a guide RNA (gRNA), wherein at least a portion of the gRNA is complementary to the target nucleic acid; then applying target nucleic acids and gRNA to a solid support to bind directly or indirectly to the solid support; then labeling the target nucleic acids; and then applying a deactivated Cas enzyme, wherein the gRNA and the Cas enzyme form a complex.

In some embodiments, the target nucleic acid is labeled before being applied to a microarray. In some embodiments, the target nucleic acid is labeled after being applied to a microarray. In some embodiments, the target nucleic acid is labeled before it is contacted with a Cas enzyme. In some embodiments, the target nucleic acid is labeled after it is contacted with a Cas enzyme.

In some embodiments, the target nucleic acid is not labeled. In some embodiments, the method comprises applying a labeled complementary nucleic acid, wherein the labeled complementary nucleic acid is complementary to a portion of the target nucleic acid. In some embodiments, the labeled complementary nucleic acid is detected.

In some embodiments, the method comprises incubating target nucleic acids with a guide RNA (gRNA), wherein at least a portion of the gRNA is complementary to the target nucleic acid, before being applied to a solid support.

In some embodiments, the method comprises incubating target nucleic acids with a guide RNA (gRNA), wherein at least a portion of the gRNA is complementary to the target nucleic acid, and a Cas enzyme, before being applied to a solid support.

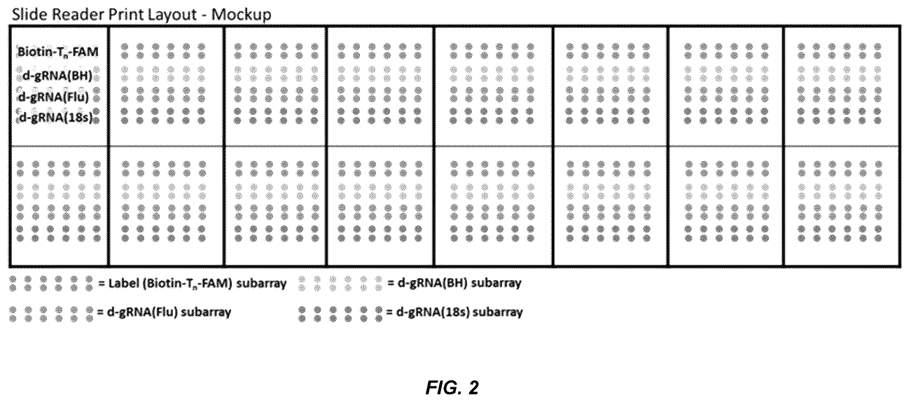

In some embodiments, the method comprises detecting multiple target nucleic acids in a sample, comprising labeling multiple target nucleic acids with a label or one or more labels, and after labeling the multiple target nucleic acids, applying the labeled target nucleic acids to a microarray comprising identifiable locations. In some embodiments, the identifiable locations are positions on a grid or pattern on a solid support, wherein the identity of the gRNA at each position is known. An example of such an array with identifiable locations is shown in FIG. 2. In some embodiments, the method comprises incubating the labeled target nucleic acids with: a first guide RNA (gRNA) bound, directly or indirectly, to a first identifiable location on a solid support, wherein at least a portion of the first gRNA is complementary to a first target nucleic acid; a second gRNA bound, directly or indirectly, to a second identifiable location on the solid support, wherein at least a portion of the second gRNA is complementary to a second target nucleic acid; optionally one or more additional gRNA bound to one or more additional identifiable locations, wherein at least a portion of the one or more additional gRNA is complementary to one or more additional target nucleic acids, respectively, and deactivated Cas enzymes, wherein each first gRNA forms a complex with a deactivated Cas enzyme and wherein each second gRNA forms a complex with a deactivated Cas enzyme. In some embodiments, the method further comprises washing the microarray. In some embodiments, the method further comprises detecting the nucleic acids by detecting the presence or absence of a signal from the identifiable locations on the microarray.

In some embodiments, the method comprises applying a first, second, and one or more additional labeled complementary nucleic acids, wherein the first labeled complementary nucleic acid is complementary to a portion of the first target nucleic acid, wherein the second labeled complementary nucleic acid is complementary to a portion of the second target nucleic acid, and wherein the one or more additional labeled complementary nucleic acids are complementary to a portion of the one or more additional target nucleic acids, respectively. In some embodiments, the labeled complementary nucleic acid is detected.

In some embodiments, methods of detecting one or more target nucleic acids in a sample comprises:
incubating the one or more target nucleic acids on a microarray comprising one or more identifiable locations with one or more guide RNAs (gRNAs) bound directly to one or more identifiable locations on a solid support, wherein at least a portion of the one or more gRNAs is complementary to the one or more target nucleic acids, respectively;
washing the microarray; and
detecting the one or more target nucleic acids by detecting a presence or an absence of a signal from the one or more identifiable locations on the microarray.

In some embodiments, methods of the present disclosure further comprise labeling the one or more target nucleic acids with a label, and after labeling the one or more target nucleic acids, applying the one or more target nucleic acids to the microarray before the incubating.

In some embodiments, methods of the present disclosure further comprise applying one or more labeled complementary nucleic acids to the microarray after the incubating and before the washing, wherein the one or more labeled complementary nucleic acids are complementary to a portion of the one or more target nucleic acids, respectively.

In some embodiments, in methods of the present disclosure, the one or more target nucleic acids are further incubated with a deactivated Cas enzyme, wherein the one or more gRNAs forms a complex with the deactivated Cas enzyme.

In some embodiments, the one or more additional gRNA bound to one or more additional identifiable locations comprise at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950 additional gRNA, at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950 additional identifiable locations, and at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950 additional targets, wherein at least a portion of the one or more additional gRNA is complementary to one or more additional targets.

In some embodiments, the method further comprises a third gRNA bound, directly or indirectly, to a third identifiable location on the solid support, wherein at least a portion of the third gRNA is complementary to a third target nucleic acid. In some embodiments, the method further comprises a fourth gRNA bound, directly or indirectly, to a fourth identifiable location on the solid support, wherein at least a portion of the fourth gRNA is complementary to a fourth target nucleic acid. In some embodiments, the method further comprises a fifth gRNA bound, directly or indirectly, to a fifth identifiable location on the solid support, wherein at least a portion of the fifth gRNA is complementary to a fifth target nucleic acid. In some embodiments, the method further comprises a sixth gRNA bound, directly or indirectly, to a sixth identifiable location on the solid support, wherein at least a portion of the sixth gRNA is complementary to a sixth target nucleic acid. In some embodiments, the method further comprises a seventh gRNA bound, directly or indirectly, to a seventh identifiable location on the solid support, wherein at least a portion of the seventh gRNA is complementary to a seventh target nucleic acid. In some embodiments, the method further comprises an eighth gRNA bound, directly or indirectly, to an eighth identifiable location on the solid support, wherein at least a portion of the eighth gRNA is complementary to an eighth target nucleic acid. In some embodiments, the method further comprises a ninth gRNA bound, directly or indirectly, to a ninth identifiable location on the solid support, wherein at least a portion of the ninth gRNA is complementary to a ninth target nucleic acid. In some embodiments, the method further comprises a tenth gRNA bound, directly or indirectly, to a tenth identifiable location on the solid support, wherein at least a portion of the tenth gRNA is complementary to a tenth target nucleic acid. In some embodiments, the method further comprises an eleventh gRNA bound, directly or indirectly, to an eleventh identifiable location on the solid support, wherein at least a portion of the eleventh gRNA is complementary to an eleventh target nucleic acid. In some embodiments, the method further comprises a twelfth gRNA bound, directly or indirectly, to a twelfth identifiable location on the solid support, wherein at least a portion of the twelfth gRNA is complementary to a twelfth target nucleic acid. In some embodiments, the method further comprises a thirteenth gRNA bound, directly or indirectly, to a thirteenth identifiable location on the solid support, wherein at least a portion of the thirteenth gRNA is complementary to a thirteenth target nucleic acid. In some embodiments, the method further comprises a fourteenth gRNA bound, directly or indirectly, to a fourteenth identifiable location on the solid support, wherein at least a portion of the fourteenth gRNA is complementary to a fourteenth target nucleic acid. In some embodiments, the method further comprises a fifteenth gRNA bound, directly or indirectly, to a fifteenth identifiable location on the solid support, wherein at least a portion of the fifteenth gRNA is complementary to a fifteenth target nucleic acid, and so on.

In some embodiments, the method further includes detecting fluorescence on the microarray using a commercial microarray scanner, for example, Innopsys® InnoScan® microarray scanner.

In some embodiments, a method of detecting a target nucleic acid in a sample, comprises:
providing a sample comprising a target nucleic acid, wherein the target nucleic acid comprises a label and is present in the sample at a concentration of about 1 nM or less;
contacting the sample with a microarray comprising a plurality of guide RNA (gRNA) molecules covalently bound to a solid support, for a time period of about 24 hours or less, wherein:
   the plurality of gRNA molecules are in a complex with a deactivated CRISPR-associated (Cas) enzyme,
   at least one gRNA molecule in the plurality comprises a nucleotide sequence that is complementary to a portion of the target nucleic acid in the sample, and
   the contacting is under conditions in which the target nucleic acid hybridizes to the complementary gRNA molecule on the apparatus;
washing the apparatus to remove unhybridized nucleic acids; and
detecting the target nucleic acid using the label.

In some embodiments, the target nucleic acid is present in the sample at a concentration of about 100 pM or less. In some embodiments, the sample is contacted with the apparatus for a time period of about 1 hour or less. In some embodiments, the deactivated Cas enzyme is deactivated Cas13 enzyme. In some embodiments, the label comprises a quantum dot.

### A. Cas Enzymes and Guide RNAs

In some embodiments, the Cas enzyme is Cas13. In some embodiments, the Cas enzyme is Cas12. In some embodiments, the Cas enzyme is Cas9. In some embodiments, the Cas enzyme is Cas14.

In some embodiments, the Cas enzyme cuts or nicks or cleaves RNA. Cas enzymes that cut or nick or cleave RNA include Cas13. In some embodiments, the Cas enzyme cuts or nicks or cleaves DNA. Cas enzymes that cut or nick or cleave DNA include Cas9, Cas12, and Cas14. In some embodiments, a Cas enzyme that cuts or nicks or cleaves RNA is chosen when the target nucleic acid is RNA. In some embodiments, a Cas enzyme that cuts or nicks or cleaves DNA is chosen when the target nucleic acid is DNA.

In some embodiments, the Cas enzyme is deactivated. In some embodiments, deactivated Cas enzymes do not cut or nick or cleave the target nucleic acid. In some embodiments, the Cas enzyme is a deactivated Cas13 enzyme. In some embodiments, the Cas enzyme is a deactivated Cas12 enzyme. In some embodiments, the Cas enzyme is a deactivated Cas9 enzyme. In some embodiments, the Cas enzyme is a deactivated Cas14 enzyme.

There are different ways of deactivating Cas enzymes, and multiple methods of deactivating Cas enzymes work for use in the various inventions described herein.

In some embodiments, the deactivated Cas enzyme is deactivated using deactivating guide RNA (d-gRNA). In some embodiments, the guide RNA sequence is complementary to a target nucleic acid, but not sufficiently complementary to allow Cas to start cleavage (or nicking or cutting) of the target nucleic acid. In some embodiments, the Cas enzyme is a “live Cas” that, if not for the deactivating guide RNA, would otherwise be capable of cleaving the target nucleic acid. Methods of using a gRNA to deactivate a Cas enzyme are described in Tambe, A. et al., “RNA Binding and HEPN-Nuclease Activation Are Decoupled in CRISPR-Cas13a,” Cell Reports 24:1025-1036 (2018), which is incorporated herein by reference for its description of methods of using a gRNA to deactivate a Cas enzyme. In some embodiments, the Cas enzyme is a live Cas13 enzyme that is then deactivated using deactivating gRNA.

In some embodiments, the deactivated Cas enzyme comprises one or more mutations that silence nucleic acid cleavage or cutting or nicking activity. In other words, the Cas enzyme itself is mutated so that it cannot cleave or nick or cut a target nucleic acid. Cas enzymes deactivated in this way are sometimes referred to as “dead Cas.” Methods of using dead Cas are described in Guk, K. et al., “A facile, rapid and sensitive detection of MRSA using a CRISPR-mediated DNA FISH method, antibody-like dCas9/sgRNA complex,” Biosensors and Bioelectronics 95:67-71 (2017), and Hajian, R. et al, “Detection of unamplified target genes via CRISPR-Cas9 immobilized on a graphene field-effect transistor,” Nature Biomedical Engineering 3:427-437 (2019), both of which are incorporated herein by reference for their description of methods of using dead Cas enzymes. In some embodiments, the deactivated Cas enzyme is “dCas13” that can bind target RNA but cannot cleave or nick or cut the target RNA. In some embodiments, dCas13 is commercially available from MCLAB®. In some embodiments, the Cas enzyme is provided in a reaction buffer.

In some embodiments, the Cas enzyme is targeted to a target nucleic acid using a guide RNA (gRNA). In some embodiments, at least a portion of the gRNA is complementary to the target nucleic acid. In some embodiments, the gRNA is completely complementary to the target nucleic acid. In some embodiments, the gRNA is not completely complementary to the target nucleic acid. In some embodiments, when the gRNA is completely complementary to the target nucleic acid, a “dead Cas” enzyme is used. In some embodiments, when the gRNA is not sufficiently complementary to the target nucleic acid to allow Cas to start cleavage of the target nucleic acid, a “live Cas” enzyme is used.

In some embodiments, the Cas enzyme is provided in solution (also referred to herein as a reaction buffer). In some circumstances, Cas enzymes are not stable in solution and tend to precipitate out of solution quite easily. In some embodiments, an objective is to provide sufficient amounts of Cas enzymes in solution to attach to guide RNAs immobilized on the surface of a solid support to make a microarray. In some embodiments, the concentration of Cas enzymes in solution is 10-25 nM, 11-25 nM, 12-25 nM, 13-25 nM, 14-25 nM, 15-25 nM, 16-25 nM, 17-25 nM, 18-25 nM, 19-25 nM, 20-25 nM, 21-25 nM, 22-25 nM, 23-25 nM, 24-25 nM, 10-24 nM, 10-23 nM, 10-22 nM, 10-21 nM, 10-20 nM, 10-19 nM, 10-18 nM, 10-17 nM, 10-16 nM, 10-15 nM, 10-14 nM, 10-13 nM, 10-12 nM, 10-11 nM, 11 nM, 12 nM, 13 nM, 14 nM, 15 nM, 16 nM, 17 nM, 18 nM, 19 nM, 20 nM, 21 nM, 22 nM, 23 nM, 24 nM, or 25 nM. In some embodiments, the solution in which the Cas enzymes are dissolved comprises HEPES, MgCl₂, NaCl, and EDTA, at pH 6.5. In some embodiments, the solution in which the Cas enzymes are dissolved comprises 20 mM HEPES, 5 mM MgCl₂, 0.1 M NaCl, 0.1 mM EDTA, at pH 6.5 (1× Cas13 reaction buffer).

### B. Nucleic Acid Labels

In some embodiments, target nucleic acids are labeled. Many types of nucleic labels are contemplated. One consideration in selecting a nucleic acid label is that it does not interfere with the functioning of the Cas enzyme.

In some embodiments, the nucleic acid label comprises a fluorescent molecule. In some embodiments, the nucleic acid label comprises Alexa Fluor® 488, Cy3 (Cyanine3), Cy5, FITC (fluorescein isothiocyanate), Texas Red, Rhodamine, FAM (fluorescein amidite), TAMRA (tetramethylrhodamine), or DAPI (4′,6-diamidino-2-phenylindole). In some embodiments, the nucleic acid label comprises Alexa Fluor® 488 (“AF488”). General categories of fluorescent labels include organic dyes, biological fluorophores, quantum dots, and nanoparticles including carbon dots. Specific fluorescent dyes include fluorescein, rhodamine, cyanine dyes, ALEXA dyes, DYLIGHT dyes, and ATTO dyes.

In some embodiments, the nucleic acid label comprises biotin.

In some embodiments, the nucleic acid label comprises a quantum dot. In some embodiments, the quantum dots are semiconductor particles a few nanometers in size and when they are illuminated by UV light, they will emit light-fluorescing crystals 1-5 nm in diameter that are excitable by a large range of wavelengths of light. These crystals emit light such as monochromatic light, with a wavelength dependent on their chemical composition and size. Quantum dots include PbS, PbS₂, PbSe, CdS, CdSe, CdTe, ZnS, ZnSe, InP, InAs, Si, Ge, and others. In some embodiments, the quantum dots are streptavidin functional quantum dots. In some embodiments, the quantum dots comprise a CdSe core and ZnS shell. In some embodiments, the quantum dots have a high concentration of Streptavidin conjugated to their surfaces. In some embodiments, the quantum dots are Qdot™ 525 Streptavidin Conjugate quantum dots. In some embodiments, the quantum dots are Qdot™ 565 Streptavidin Conjugate quantum dots. In some embodiments, the quantum dots are Qdot™ 585 Streptavidin Conjugate quantum dots. In some embodiments, the quantum dots are Qdot™ 605 Streptavidin Conjugate quantum dots. In some embodiments, the quantum dots are Qdot™ 625 Streptavidin Conjugate quantum dots. In some embodiments, the quantum dots are Qdot™ 655 Streptavidin Conjugate quantum dots. In some embodiments, the quantum dots are Qdot™ 705 Streptavidin Conjugate quantum dots. In some embodiments, the quantum dots are Qdot™ 800 Streptavidin Conjugate quantum dots. In some embodiments, the quantum dots emit at 525 nm, 565 nm, 585 nm, 605 nm, 625 nm, 655 nm, 705 nm, or 800 nm.

Other potential labels include a colorimetric reagent, a chromogenic molecule or protein, a Raman label, a chromophore, or gold or silver particles.

In some embodiments, the nucleic acid label is another nucleic acid that hybridizes to the target nucleic acid and then extends at its ends with labeled nucleotides.

In some embodiments, nucleic acid labels are covalently attached to the target nucleic acid. Many options are available for attaching nucleic acid labels to the target nucleic acid, including various labeling kits and linkers. Examples of such linkers include NHS (N-hydroxysuccinimide)-based linkers and amine-reactive linkers like EDC (1-ethyl-3-(3-dimethylaminopropyl) carbodiimide) and sulfo-NHS (N-hydroxysulfosuccinimide).

In some embodiments, the label is attached to the target nucleic acid using a labeling kit. Many different labeling kits are available. In some embodiments, the label is attached to the target nucleic acid using a Mirus® linker. In some embodiments, a Mirus® labeling method is used, wherein the Mirus® linker comprises a quaternary amine, which may help facilitate subsequent hybridization to the labeled target nucleic acid. In some embodiments, the Mirus® labeling method is the Mirus® Label IT® Nucleic Acid Labeling Kit.

The Mirus® labeling method allows one to label any DNA or RNA template and is suitable for a wide range of applications. It is a one-step chemical method. It is possible to adjust the labeling density. The Mirus® labeling method is a covalent mechanism that is a permanent, non-destructive modification of nucleic acid residues. The hybridization performance of the labeled nucleic acids is not impacted by the Mirus® labeling method. One can use the Mirus® labeling method to attach different fluorophores with distinct excitation and emission spectra. In some embodiments, the Mirus® labeling method attaches AF488 to a target nucleic acid. In some embodiments, the Mirus® labeling method attaches biotin to a target nucleic acid. The Mirus® labeling method comprises a Mirus® reagent comprising a label, a linker that facilitates electrostatic interactions with nucleic acids, and a reactive alkylating group that covalently attaches to any reactive heteratom with the nucleic acid. A chemical structure of a Mirus® reagent is shown below:

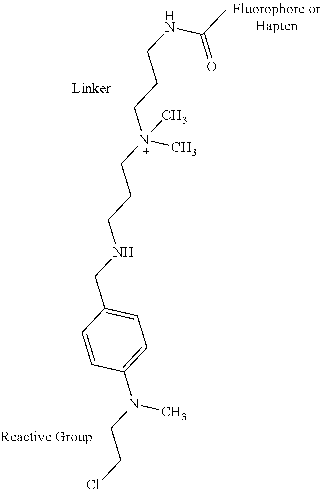 [SMILES: CC(=O)NCCC[N+](C)(C)CCCNCc1ccc(N(C)CCCl)cc1]

In some embodiments, a Mirus® linker comprises a linker that facilitates electrostatic interactions with nucleic acids, and a reactive alkylating group that covalently attaches to any reactive heteratom with the nucleic acid.

In some embodiments, the nucleic acid label is attached to the target nucleic acid using a linker. In some embodiments, the linker that attaches nucleic acid labels to the target nucleic acid incorporates a quaternary amine.

In some embodiments, a PHOTOPROBE® Biotin for Nucleic Acid Labeling kit is used to attach nucleic acid labels to the target nucleic acid. In some embodiments, a Vector Laboratories 3′ EndTag™ DNA End Labeling System is used to attach nucleic acid labels to the target nucleic acid. In some embodiments, an Applied Biosystems™ FlashTag™ Biotin HSR RNA labeling kit is used to attach nucleic acid labels to the target nucleic acid. In some embodiments, an Invitrogen Ulysis™ nucleic acid labeling kit is used to attach nucleic acid labels to the target nucleic acid. Other potential approaches include the Thermo Scientific™ EZ-Link™ Sulfo-NHS-Biotin kit or the Thermo Scientific™ EZ-Link™ PFP-Biotin kit.

In some embodiments, the target nucleic acids are labeled before applying the target nucleic acid to a microarray. In some embodiments, the target nucleic acids are labeled after applying the target nucleic acids to a microarray.

In some embodiments, providing a corresponding amount of labeling reagent to the target nucleic acid can enhance the method. In some embodiments, the target nucleic acid has about: one label every 20-60 base pairs, one label every 20-55 base pairs, one label every 20-50 base pairs, one label every 20-45 base pairs, one label every 20-40 base pairs, one label every 20-35 base pairs, one label every 20-30 base pairs, one label every 20-25 base pairs, one label every 25-60 base pairs, one label every 30-60 base pairs, one label every 35-60 base pairs, one label every 40-60 base pairs, one label every 45-60 base pairs, one label every 50-60 base pairs, or one label every 55-60 base pairs. In some embodiments, the target nucleic acid has about: one label every 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 base pairs. Using up the labeling reagent in labeling the target can prevent any unwanted interference with the Cas enzyme's process or gRNAs. In some embodiments in which Mirus® reagent is used, a solution comprising the target nucleic acid and the Mirus® reagent (“the labeling reaction”) is passed through a size selective affinity columns or columns. In some embodiments, the size selective affinity column is a G-50 column. In some embodiments, the size selective affinity column is a Zeba™ column. In some embodiments in which Mirus® reagent is used, the concentration of target nucleic acid in the labeling reaction is greater than about 0.5 mg/mL. In some embodiments in which Mirus® reagent is used, the amount of Mirus® reagent used is reduced so that less Mirus® reagent is left over after the labeling reactions.

C. Guide RNAs (gRNAs) Bound to Solid Supports

In some embodiments, guide RNAs (gRNAs) are bound, directly or indirectly, to a solid support. In some embodiments, gRNAs are bound directly to a solid support. In some embodiments, the solid support, e.g., a glass slide, is globally functionalized with epoxy silane, e.g., (3-Glycidyloxypropyl) trimethoxysilane. In some embodiments, 5′ amine functional gRNA oligos are dispensed in buffer (e.g., 3×SSC) onto the epoxy silane functionalized solid support, e.g., epoxy silane functionalized glass. In some embodiments, the solid supports are incubated in high humidity (e.g., about 90%) to allow printed 5′ amines to ring open the epoxy groups and covalently anchor the oligos. In some embodiments, the solid supports are washed in a series of buffers to remove excess oligos. In some embodiments, residual surface epoxy groups are quenched with small amine molecules such as ethanolamine. In some embodiments, the gRNAs are dispensed onto the solid support using a dispensing system. In some embodiments, the dispensing system is a printer. In some embodiments, the printer is a BioDot printer.

In some embodiments, the gRNAs are bound to the solid support via amine/epoxy coupling. In some embodiments, the gRNA is 5′ amine modified.

In some embodiments, the guide sequence is 20 base pairs in length. In some embodiments, the guide sequence is 15 base pairs in length. In some embodiments, the guide sequence is 16 base pairs in length. In some embodiments, the guide sequence is 17 base pairs in length. In some embodiments, the guide sequence is 18 base pairs in length. In some embodiments, the guide sequence is 19 base pairs in length. In some embodiments, the guide sequence is 21 base pairs in length. In some embodiments, the guide sequence is 22 base pairs in length. In some embodiments, the guide sequence is 23 base pairs in length. In some embodiments, the guide sequence is 24 base pairs in length. In some embodiments, the guide sequence is 25 base pairs in length.

In some embodiments, the gRNAs are printed onto a solid support in a predetermined pattern to create identifiable locations. For example, FIG. 2 shows a pattern for four different types of gRNAs.

In some embodiments, the gRNAs to be printed on the solid support are present in the print buffer at a concentration of about 10-40 μM, 10-35 μM, 10-30 μM, 10-25 μM, 10-20 μM, 10-15 μM, 15-40 μM, 20-40 μM, 25-40 μM, 30-40 μM, or 35-40 μM. In some embodiments, the gRNAs to be printed on the solid support are present in the print buffer at a concentration of about 10 μM, 11 μM, 12 μM, 13 μM, 14 μM, 15 μM, 16 μM, 17 μM, 18 UM, 19 μM, 20 μM, 21 μM, 22 μM, 23 μM, 24 UM, 25 μM, 26 μM, 27 UM, 28 UM, 29 μM, 30 μM, 31 μM, 32 μM, 33 μM, 34 μM, 35 μM, 36 μM, 37 μM, 38 μM, 39 μM, or 40 μM.

In some embodiments, the degree of complementarity or identity between a guide sequence and its corresponding target sequence may be about 75%, 80%, 85%, 90%, 91%, 92%, 93%, 94%, 95%, 96%, 97%, 98%, 99%, or 100%. In some embodiments, the guide sequence and the target region may be 100% complementary or identical. In some embodiments, the guide sequence and the target region may be 99% complementary or identical. In some embodiments, the guide sequence and the target region may be 98% complementary or identical. In some embodiments, the guide sequence and the target region may be 97% complementary or identical. In some embodiments, the guide sequence and the target region may be 96% complementary or identical. In some embodiments, the guide sequence and the target region may be 95% complementary or identical. In some embodiments, the guide sequence and the target region may be 94% complementary or identical. In some embodiments, the guide sequence and the target region may be 93% complementary or identical. In some embodiments, the guide sequence and the target region may be 92% complementary or identical. In some embodiments, the guide sequence and the target region may be 91% complementary or identical. In some embodiments, the guide sequence and the target region may be 90% complementary or identical. In some embodiments, the guide sequence and the target region may be 85% complementary or identical. In some embodiments, the guide sequence and the target region may be 80% complementary or identical. In some embodiments, the guide sequence and the target region may be 75% complementary or identical.

In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

In some embodiments, the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the protospacer adjacent motif (PAM)) except for the substitution of U for T in the guide sequence.

### D. Target Nucleic Acids

In some embodiments, the target nucleic acid is deoxyribonucleic acid (DNA). In some embodiments, the target nucleic acid is ribonucleic acid (RNA).

In some embodiments, the target nucleic acid is genomic. In some embodiments, the target nucleic acid is synthetic. In some embodiments, the target nucleic acid is genomic DNA. In some embodiments, the target nucleic acid is synthetic DNA. In some embodiments, the target nucleic acid is extrachromosomal DNA. In some embodiments, the target nucleic acid is extracellular DNA. In some embodiments, the target nucleic acid is genomic RNA. In some embodiments, the target nucleic acid is messenger RNA (mRNA). In some embodiments, the target nucleic acid is noncoding RNA (e.g., miRNA, lincRNA). In some embodiments, the target nucleic acid is transfer RNA (tRNA). In some embodiments, the target nucleic acid is synthetic RNA.

In some embodiments, the target nucleic acid is from a bacterium. In some embodiments, the target nucleic acid is from a virus. In some embodiments, the target nucleic acid is from a eukaryote, optionally from a mammal, further optionally from a human.

In some embodiments, the target nucleic acid is isolated. In some embodiments, the target nucleic acid is not isolated.

### E. Solid Supports

In some embodiments, the methods of detecting a target nucleic acid or multiple target nucleic acids, microarrays, and/or kits for the same comprise use of a solid support.

In some embodiments, solid supports may include known materials, such as slides, chips, and particles free in solution, which may be made of nitrocellulose, nylon, glass, epoxy, one or more polymers, or other compositions, of which one embodiment is magnetically attractable particles.

In some embodiments, the methods of detecting a target nucleic acid or multiple target nucleic acids and/or kits for the same comprise use of a solid support, wherein the solid support is a slide. In some embodiments, the slide comprises an epoxy slide.

In some embodiments, the solid support comprises glass, a semiconductor, a metal, carbon, a polymer, cellulose, a metal oxide, or a combination thereof. In some embodiments, the solid support comprises glass. In some embodiments, the solid support comprises a semiconductor (e.g., silicon, silicon nitride), metal (e.g., gold, platinum), carbon (graphite, graphene), a polymer (e.g., polycarbonate), cellulose, and/or a metal oxide (e.g., ITO, TiO2).

### F. Blocking Solutions

In some embodiments, a blocking solution is used. In some embodiments, the apparatus is incubated with a blocking solution prior to detecting the target nucleic acid. In some embodiments, the blocking solution is used to prevent nonspecific binding of the Cas enzyme to the surface of the solid support and/or to prevent RNase from degrading the immobilized gRNA printed on the solid support. It can be undesirable to allow the Cas enzymes to non-specifically bind across the entire surface of the solid support, forming a film.

In some embodiments, the blocking solution is free of, or nearly free of, RNase activity. In some embodiments, the RNase activity in the blocking solution has been neutralized or reduced.

In some embodiments, the blocking solution comprises a protein blocker or a non-protein blocker. In some embodiments, the blocking solution comprises a non-protein blocker. In some embodiments, the blocking solution comprises NAP (Non-Animal Protein)-BLOCKER™, Intercept® Blocking Buffer, or Pierce™ Non-Protein Blocker. In some embodiments, the non-protein blocker is Pierce™ Non-Protein Blocker.

In some embodiments, the blocking solution comprises a protein blocker. In some embodiments, the blocking solution comprises Denhardt's Solution, casein, bovine serum albumin, gelatin, non-fat dry milk, or Thermo Scientific™ SuperBlock™ blocking buffer. In some embodiments, the blocking solution comprises Thermo Scientific™ SuperBlock™ blocking buffer. In some embodiments, the blocking solution comprises RNAᵢ. In some embodiments, the blocking solution comprises RNase inhibitor. In some embodiments, the blocking solution comprises Invitrogen™ SUPERase·In™ RNase inhibitor.

### III. Microarrays

The present disclosure also relates to microarrays for identifying the presence of one or more target nucleic acids, the microarrays comprising: one or more identifiable locations on a solid support; and one or more guide RNAs (gRNAs) bound (e.g., bound directly or indirectly) to the one or more identifiable locations on the solid support, wherein at least a portion of the one or more gRNAs is complementary to the one or more target nucleic acids, respectively.

In some embodiments, a microarray comprises a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid.

In some embodiments, microarrays of the present disclosure comprise gRNAs bound to the solid support via covalent coupling (e.g., amine/epoxy coupling). In some embodiments, the gRNA is 5′ amine modified. In some embodiments, the solid support comprises glass, a semiconductor, a metal, carbon, a polymer, cellulose, a metal oxide, or a combination thereof. In some embodiments, the plurality of gRNA molecules on the apparatus are modified. In some embodiments, the plurality of gRNA molecules on the apparatus are 5′ amine-modified.

In some embodiments, the plurality of gRNA molecules on the apparatus are in a complex with a CRISPR-associated (Cas) enzyme. In some embodiments, the Cas enzyme is a deactivated Cas enzyme. In some embodiments, the deactivated Cas enzyme is a deactivated Cas9 enzyme, a deactivated Cas12 enzyme, or a deactivated Cas13 enzyme. In some embodiments, the deactivated Cas enzyme is deactivated Cas13 enzyme.

### IV. Kits

Also disclosed herein are kits comprising: a microarray comprising a plurality of nucleic acid (e.g., guide RNA (gRNA)) molecules bound, directly or indirectly, to a solid support. In some embodiments, the microarray comprises multiple identifiable locations, wherein each identifiable location comprises a nucleic acid (e.g., gRNA) molecule having a nucleotide sequence that is complementary to the nucleotide sequence of at least one target nucleic acid.

In some embodiments, a kit comprises: a microarray comprising a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid. In some embodiments, the solid support comprises glass, a semiconductor, a metal, carbon, a polymer, cellulose, a metal oxide, or a combination thereof. In some embodiments, the plurality of gRNA molecules on the apparatus are modified. In some embodiments, the plurality of gRNA molecules on the apparatus are 5′ amine-modified.

In some embodiments, the plurality of gRNA molecules on the apparatus are in a complex with a CRISPR-associated (Cas) enzyme. In some embodiments, the Cas enzyme is a deactivated Cas enzyme. In some embodiments, the deactivated Cas enzyme is a deactivated Cas9 enzyme, a deactivated Cas12 enzyme, or a deactivated Cas13 enzyme. In some embodiments, the deactivated Cas enzyme is deactivated Cas13 enzyme. In some embodiments, kits of the present disclosure further comprise one or more of: a nucleic acid label, a blocking solution, a labeled oligonucleotide, a reagent for attaching a nucleic acid label to a target nucleic acid, and a Cas enzyme.

In some embodiments, the kit comprises a microarray comprising a guide RNA (gRNA) bound (e.g., bound directly or indirectly) to a solid support. In some embodiments, the gRNA is bound to the solid support via amine/epoxy coupling, wherein the gRNA is 5′ amine-modified, further wherein at least a portion of the gRNA is complementary to a target nucleic acid. In some embodiments, kits of the present disclosure further comprise a deactivated Cas enzyme; a nucleic acid label; blocking solution; or a combination thereof.

In some embodiments, the kit comprises a microarray comprising: a first guide RNA (gRNA) bound to a solid support via amine/epoxy coupling to a first identifiable location on the microarray, wherein the first gRNA is 5′ amine-modified, further wherein at least a portion of the first gRNA is complementary to a first target nucleic acid; a second guide RNA (gRNA) bound to the solid support via amine/epoxy coupling to a second identifiable location on the microarray, wherein the first gRNA is 5′ amine-modified, further wherein at least a portion of the second gRNA is complementary to a second target nucleic acid; optionally one more additional guide RNA (gRNA) bound to the solid support via amine/epoxy coupling to one or more additional identifiable locations on the microarray, wherein the one or more additional gRNAs are 5′ amine-modified, further wherein at least a portion of the one or more additional gRNAs are complementary to one or more additional target nucleic acids, respectively; a deactivated Cas enzyme; a nucleic acid label; and blocking solution.

In some embodiments, the kit comprises at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950 additional gRNA, at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950, additional identifiable locations, and at least about 3-1000, 3-100, 3-75, 3-50, 3-25, 3-15, 3-10, 250-1000, 300-1000, 350-1000, 400-1000, 450-1000, 500-1000, 550-1000, 600-1000, 650-1000, 700-1000, 750-1000, 800-1000, 850-1000, 900-1000, 950-1000, 250-300, 250-350, 250-400, 250-450, 250-500, 250-550, 250-600, 250-650, 250-700, 250-750, 250-800, 250-850, 250-900, or 250-950 additional targets.

In some embodiments, the kit further comprises a third gRNA bound to a third identifiable location on the solid support, wherein at least a portion of the third gRNA is complementary to a third target nucleic acid. In some embodiments, the kit further comprises a fourth gRNA bound to a fourth identifiable location on the solid support, wherein at least a portion of the fourth gRNA is complementary to a fourth target nucleic acid. In some embodiments, the kit further comprises a fifth gRNA bound to a fifth identifiable location on the solid support, wherein at least a portion of the fifth gRNA is complementary to a fifth target nucleic acid. In some embodiments, the kit further comprises a sixth gRNA bound to a sixth identifiable location on the solid support, wherein at least a portion of the sixth gRNA is complementary to a sixth target nucleic acid. In some embodiments, the kit further comprises a seventh gRNA bound to a seventh identifiable location on the solid support, wherein at least a portion of the seventh gRNA is complementary to a seventh target nucleic acid. In some embodiments, the kit further comprises an eighth gRNA bound to an eighth identifiable location on the solid support, wherein at least a portion of the eighth gRNA is complementary to an eighth target nucleic acid. In some embodiments, the kit further comprises a ninth gRNA bound to a ninth identifiable location on the solid support, wherein at least a portion of the ninth gRNA is complementary to a ninth target nucleic acid. In some embodiments, the kit further comprises a tenth gRNA bound to a tenth identifiable location on the solid support, wherein at least a portion of the tenth gRNA is complementary to a tenth target nucleic acid. In some embodiments, the kit further comprises an eleventh gRNA bound to an eleventh identifiable location on the solid support, wherein at least a portion of the eleventh gRNA is complementary to an eleventh target nucleic acid. In some embodiments, the kit further comprises a twelfth gRNA bound to a twelfth identifiable location on the solid support, wherein at least a portion of the twelfth gRNA is complementary to a twelfth target nucleic acid. In some embodiments, the kit further comprises a thirteenth gRNA bound to a thirteenth identifiable location on the solid support, wherein at least a portion of the thirteenth gRNA is complementary to a thirteenth target nucleic acid. In some embodiments, the kit further comprises a fourteenth gRNA bound to a fourteenth identifiable location on the solid support, wherein at least a portion of the fourteenth gRNA is complementary to a fourteenth target nucleic acid. In some embodiments, the kit further comprises a fifteenth gRNA bound to a fifteenth identifiable location on the solid support, wherein at least a portion of the fifteenth gRNA is complementary to a fifteenth target nucleic acid.

In some embodiments, the kit further comprises one or more of: a nucleic acid label, a blocking solution, a labeled oligonucleotide, a reagent for attaching a nucleic acid label to a target nucleic acid, and a Cas enzyme.

In some embodiments, the kit comprises a Cas enzyme in solution.

In some embodiments, the kit comprises a Cas enzyme that has not yet been deactivated, and will become deactivated upon forming of a complex with a deactivating gRNA.

In some embodiments, the kit further comprises a reagent for attaching the nucleic acid label to the target nucleic acid. In some embodiments, the reagent is Mirus® reagent. Mirus® reagent includes the label itself as well as Mirus® linker comprising a linker that facilitates electrostatic interactions with nucleic acids and a reactive alkylating group that covalently attaches to any reactive heteroatom with the nucleic acid.

In some embodiments, the kit further comprises a labeled complementary nucleic acid, wherein the labeled complementary nucleic acid is complementary to a portion of the target nucleic acid. In some embodiments, the kit further comprises a first second, and one or more additional labeled complementary nucleic acids, wherein the first labeled complementary nucleic acid is complementary to a portion of the first target nucleic acid, wherein the second labeled complementary nucleic acid is complementary to a portion of the second target nucleic acid, and wherein the one or more additional labeled complementary nucleic acids are complementary to a portion of the one or more additional target nucleic acids, respectively.

### EXAMPLES

### Example 1. Assay Preparation

Example 1 describes an example assay protocol that was followed for preparing and running assays described herein.

### A. Materials

The following materials were used to prepare and run an assay.
Epoxy slides with printed microarrays of controls/gRNAs
Grace Bio-Labs ProPlate® Multi-Well chambers (16 wells)
Mirus® Label IT® kit
   Label IT® AF488 Reagent (reconstituted with Reconstitution Solution provided in kit)
   10× Labeling Buffer A (provided in kit)
Target solutions (synthetic or genomic; for example, BH, 18s, FluA)
Invitrogen™ SUPERase·In™ RNase inhibitor
Thermo Scientific™ Zeba™ desalting columns (2 mL, 7 k MWCO)
10× MCLAB® Cas13 reaction buffer. 0.1% Triton™ X
1 mM HCl
100 mM KCl.
50 mM ethanolamine in 0.1 M Tris (pH 9)
Thermo Scientific™ SuperBlock™ (T20 (TBS))
dCas13
3× saline-sodium citrate (SSC)

### B. Mirus® Labeling and Target Solution Preparation

Mirus® reagent was thawed at room temperature and target solutions were thawed on ice. When synthetic target was used, 5 μL of a 1 mg/mL solution was prepared by diluting in ultrapure-H₂O. When genomic target was used, 5 μL was drawn directly from extracted stock solution. 5 μL target solution was added to 35 μL of UP-H₂O. Solution was heated at 65° C. for 10 minutes to denature RNA, then quenched on ice immediately. 5 μL of 10× Labeling Buffer A was added to the target solution. 5 μL of the Mirus® Label. It® reagent was added last. It was vortexed briefly to mix. The solutions were incubated at 37° C. for 3 h and protected from light by wrapping tube(s) in aluminum foil. The reaction solution was quenched on ice. 650 μL of UP-H₂O was added and the reaction solution was vortexed briefly to mix. Labeled target was isolated with 2 mL Zeba™ desalting columns (7 k MWCO). Isolated labeled target was diluted to desired concentrations with ultrapure-H₂O and 10× MCLAB Cas13 reaction buffer+RNAᵢ (10 μL/mL). The highest concentration that can be obtained is 0.9× of the concentration off the column, as the buffer needs to be diluted to the desired 1× MCLAB® Cas13 reaction buffer+RNAᵢ (1 μL/mL).

### C. Slide Quenching, Washing, and Blocking

gRNAs/control oligomers were first deposited onto an epoxy slide. After immobilizing and curing the gRNAs/control oligomers onto the slide, the slide was bulk washed in a 50 ml conical with 0.1% Triton™ X solution on a rocker for 5 minutes. The tube was protected from light by wrapping it in aluminum foil for all bulk washes. The slide was bulk washed with 1 mM HCl for 2 minutes on a rocker. This was repeated with an additional 1 mM HCl wash for 2 minutes. The slide was bulk washed with 100 mM KCl for 10 minutes on a rocker. The slide was bulk washed with Milli-Q® H₂O for 1 minute on a rocker. The slide was bulk washed with 50 mM ethanolamine in 0.1 M Tris (pH 9) for 15 minutes on a rocker. The slide was bulk washed with Milli-Q® H₂O for 1 minute on a rocker. The slide was dried by spinning at 500 ref for 2 minutes. Grace Bio-Labs ProPlate® Multi-Well chambers were applied to the slide to hold fluid over each printed microarray. The slide was treated with 100 μL SuperBlock™+RNAᵢ (1 μL/mL) per well for at least 1 hour protected from light at room temperature.

D. dCas13 Incubation

After blocking, the slide was washed 3 times with 3×SSC+RNAᵢ (0.5 μL/mL). For Grace Bio-Labs ProPlate® Multi-Well chambers, each wash required 2 mL of solution distributed across the entire plate. 2 mL of solution were distributed equally across wells on slide, then the slide was agitated manually for approximately 10-20 seconds. Solution was flicked out into a waste receptacle and refilled for the next wash. The slide was treated with 100 μL per well of 12.5 nM dCas13 in 1× MCLAB® Cas13 reaction buffer+RNAᵢ (1 μL/mL). It was incubated for 30 minutes at 37° C. protected from light.

### E. Target Incubation

After dCas 13 treatment, the slide was washed 3 times with 3×SSC+RNAᵢ (0.5 μL/mL). The slide was treated with 100 μL per well of labeled targets at desired concentration in 1× MCLAB® Cas13 reaction buffer+RNAᵢ (1 L/mL). It was incubated for 30 minutes at 37° C. protected from light.

### F. Bulk Washing and Slide Imaging

After target incubation, the slide was washed and prepared for imaging by increasing the volume of wash solution to approximately 4 mL per wash, filling wells nearly to the top for each wash. The slide was washed 1 time each with: a) 3×SSC+RNAᵢ (0.5 μL/mL); b) 3×SSC+RNAᵢ (0.5 μL/mL); and c) 0.2×SSC+RNAᵢ (0.5 μL/mL). The slide was bulk washed with 0.2×SSC for 5 minutes on a rocker protected from light. The slide was dried by spinning at 500 rcf for 2 minutes. The slide was imaged by exciting at 488 nm and reading at 520 nm.

### Example 2. Single-Plex Method of Detecting Target Nucleic Acids

The assay was prepared as described in Example 1. The target RNA molecules were covalently labeled with a fluorescent molecule, Alexa Fluor® 488 (“AF388”). Three synthetic RNA targets were utilized for this purpose and are listed below in Table 1:

TABLE 1
Target RNA Sequences

| | Tar- | | SEQ |
|---|---|---|---|
| RNA | get | | ID |
| Target | Abbr. | RNA Target Sequence | NO: |
| Bordetella | BH | GGGCUAAGCGAUAAACCGAAGCUGCGGGUG | 1 |
| holmesii | | UGUACUAUGUACACGCGGUA | |
| H1N1 | Flu | GCCACCCCAAAAAUGAAGGGGACUAAAACA | 2 |
| FluA | | CUUCUAACCGAGGUCGAAAC | |
| 18s | 18s | GCCACCCCAAAAAUGAAGGGGACUAAAACA | 3 |
| | | UGUCCGGGCCGGGUGAGGUU | |

Detection of labeled RNA targets (RNA-AF388) using the d-gRNA/Cas13/RNA-AF488 assay was demonstrated by employing a labeling and binding process generally outlined in FIG. 1. Glass epoxy-functional slides were printed onto with 5′ amine-modified d-gRNA oligomers using a BioDot printer to define specific binding sites for each corresponding target arranged on the surface in microarrays. Cas13 enzyme was then incubated on the printed d-gRNA arrays on the surface to form active d-gRNA/Cas complexes. Concurrently, target RNA molecules were labeled with an Alexa Fluor® 488-containing Mirus® reagent. The Mirus® labeling method installs multiple fluorophores along the target backbone. After labeling, excess AF488 Mirus® reagent was filtered from the labeled target using a size selective separation column. Filtered and labeled RNA target molecules were diluted in MBuffer1 (a Cas13 reaction buffer) and incubated over d-gRNA/Cas microarrays. After target binding (i.e., d-gRNA/Cas complexes binding to labeled target) and washing, microarrays were dried and imaged using a commercial microarray scanner (Innopsys® InnoScan®).

On a single, epoxy-functional standard microscope slide, 16 microarrays were printed containing 4 subarrays as depicted in FIG. 2. The four sub-arrays include: (1) a fluorescent positive control oligomer with structure NH₂-Tₙ-FAM, (2) the d-gRNA (BH) (a bacterial pathogen, Bordetella holmesii) oligomer, (3) the d-gRNA (FluA) (a viral pathogen) oligomer, and (4) the d-gRNA (18s) (18s is a human biomarker) with several replicates of each in every sub-array. With this layout, up to 16 unique assays can be run on a single slide. A typical series of tests were run by placing the corresponding target solutions over the microarrays in wells defined by microchambers.

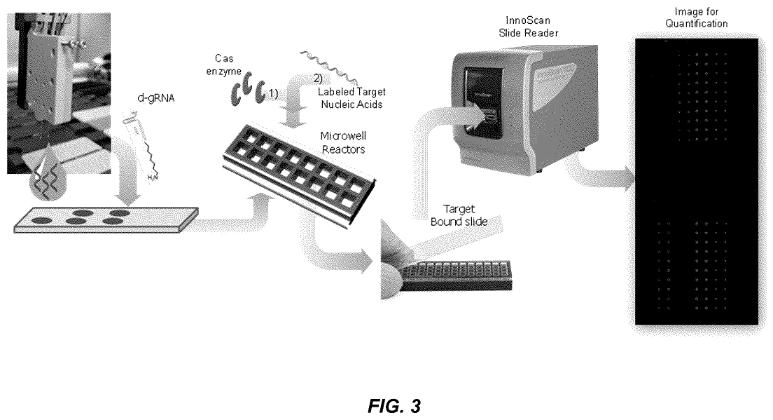

The entire workflow is illustrated in FIG. 3. First, the glass slide was globally functionalized with an epoxy silane, then 5′ amine modified deactivating guide RNA (d-gRNA) RNA is also printed onto the surface using a BioDot printer to bind with the epoxy groups via amine/epoxy coupling. Next, Cas enzymes are added to the surface to bind the d-gRNA. Microwell reactors are added on top of the surface to create reaction wells. Then, labeled target nucleic acids are added to the reaction wells. The d-gRNA/Cas complexes bind to the target nucleic acid, all of which is bound to the slide. The slide is placed into an InnoScan® 1100 Slide Reader, which provides an image for quantification of target nucleic acids.

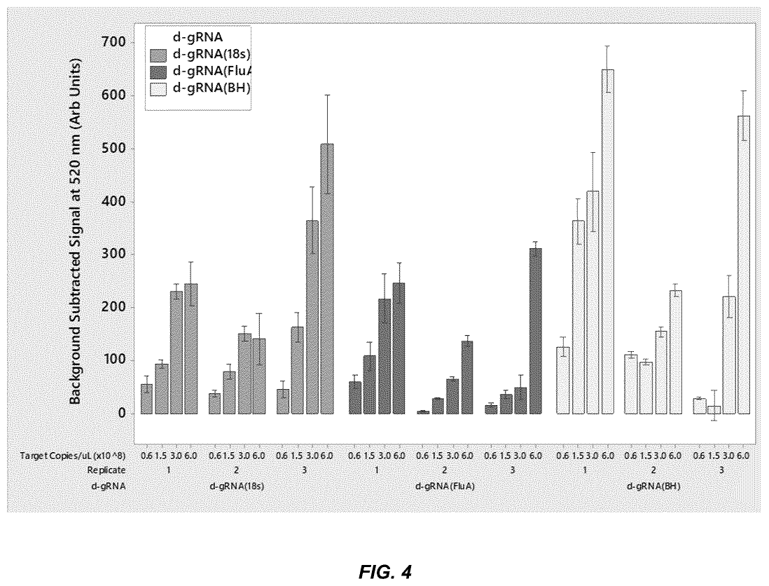

The assay was tested with three different targets: a bacterial pathogen, Bordetella holmesii (BH), a viral pathogen, FluA, and a human biomarker, 18s. Purified synthetic RNA molecules with sequences corresponding to BH, FluA, and 18s were used in the assay. These targets were evaluated individually in a concentration series and combined in multiplexing studies. For the concentration series, target binding was evaluated over a range of 6×10¹⁰ to 6×10⁹ copies/100 μL for each target, BH, FluA, and 18s. Plots for the target concentration series are shown in FIG. 4. In FIG. 4, the y-axis shows background subtracted fluorescent signal at 520 nm (Arb units) and the x-axis shows the number of copies of target nucleic acid, measured in target copies per μL (×10\hat{ }8). Here, each replicate demonstrated a general monotonic increase in signal with increasing target concentration. With the exception of BH at 1.5×10¹⁰ copies in replicate 3, all samples generated signal well above baseline. Signal from FluA was overall lower than for BH indicating a less active d-gRNA/Cas complex for this target. Still the general trend in signal matched that of BH and all samples produced signal above baseline. Notably, the 6×10⁹ sample in replicate 2 gave only marginally positive signal. Finally, signal for 18s was, again, monotonic over the concentration range evaluated. In the case of 18s, signal was intermediate between FluA and BH and all samples were clearly above baseline indicating a high consistency in binding for the d-gRNA (18s)/Cas complex.

### Example 3. Multiplex Method of Detecting Target Nucleic Acids

To evaluate the specificity of targeted sub-arrays for binding their respective targets, the d-gRNA/Cas microarrays were tested in a multiplexed format. The assay was prepared as described in Example 1. Purified synthetic RNA molecules with sequences corresponding to BH, FluA, and 18s were used in the assay. For these assessments, two levels of multiplexing were carried out, duplexing and triplexing. Duplex tests involved exposing two of the three pathogen targets to a complete microarray while omitting the third pathogen. For this capability test, the three duplex combinations are listed in Table 2.

TABLE 2
List of Duplex Target Combinations
Evaluated in Multiplexed Tests

| Duplex | |
|---|---|
| Combination | Targets |
| 1 | 18s, BH |
| 2 | 18s, Flu |
| 3 | Flu, BH |

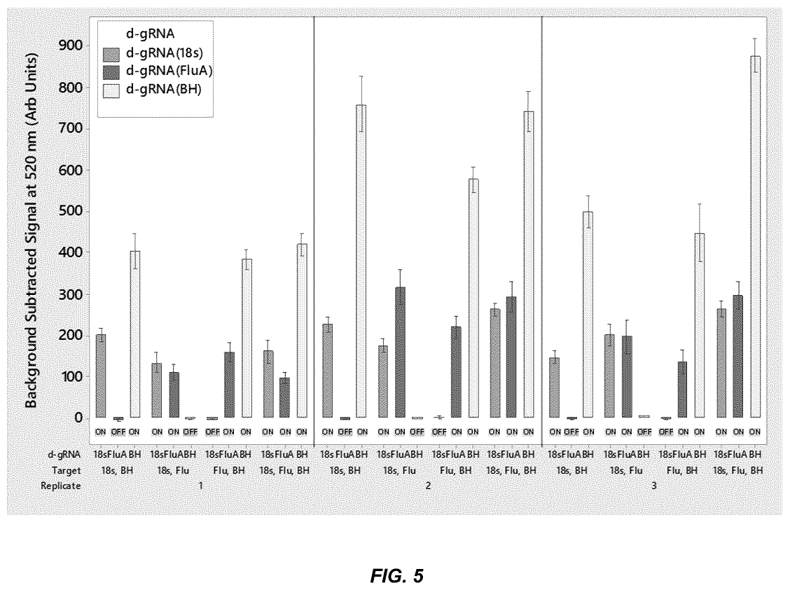

The triplex test included all three pathogens in a single solution incubated over the microarray. The concentration was 6×10⁸ copies/μL. For all multiplexing tests a strong signal over background was expected for targets present and no signal was expected over background for omitted targets. Replicates for each scenario are depicted in FIG. 5. The y-axis shows background subtracted fluorescent signal at 520 nm (Arb units). “On” indicates that the indicated target was included, and “off” indicates that the indicated target was not included.

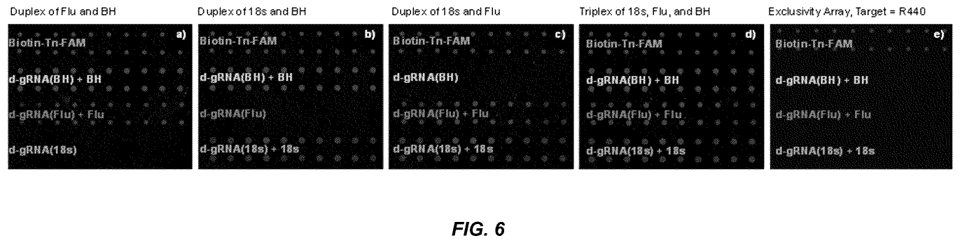

In all cases the sub-arrays for the included targets gave signal significantly above background while signal for sub-arrays without targets remained equivalent to the background. For example, in replicate 2 for duplex +18s, −Flu, +BH, strong signal was seen for 18s and BH but no signal for Flu. Furthermore, in the same replicate, the triplex, which includes 18s, Flu, and BH resulted in strong signal for all three targets. An image for duplex (a-c) and triplex (d) assays is shown in FIG. 6. Purified synthetic RNA molecules with sequences corresponding to BH, FluA, and 18s were used in the assay. Each of panels (a)-(c) in FIG. 6 includes Biotin-Tn-FAM as a control. Each panel is a microarray with d-gRNA/Cas for Biotin-Tn-FAM, BH, FluA, and 18s. In each panel, the microarray was fabricated by printing biotinylated oligonucleotides on streptavidin functional glass such that the oligonucleotides were anchored to the substrate through biotin/streptavidin binding. Panel (a) in FIG. 6 shows a fluorescent image after sample with FluA and BH was applied to the microarray. Panel (b) in FIG. 6 shows a fluorescent image after sample with 18s and BH was applied to the microarray. Panel (c) in FIG. 6 shows a fluorescent image after sample with 18s and FluA was applied to the microarray. Panel (d) in FIG. 6 shows a fluorescent image after sample with BH, FluA, and 18s was applied to the microarray. Panel (c) in FIG. 6 shows a fluorescent image after sample with R440, a sequence from Chlamydia, was applied to the microarray (to test off-target binding).

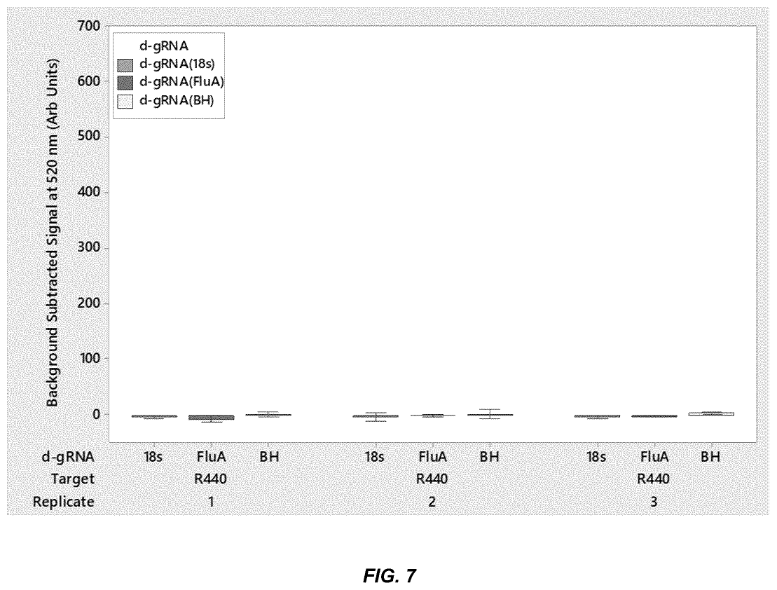

Off panel target binding or exclusivity was also evaluated. The intent was to assess the degree of binding from an off-panel target to the on-panel sub-arrays. The target molecule used for this purpose was a sequence from Chlamydia known as R440 at a concentration of 6×10⁸ copies/μL. When exposed to the full microarray, R440 did not bind to any sub-array on the surface as evidenced by baseline signal from all replicates as shown in FIG. 7. In FIG. 7, the y-axis shows background subtracted fluorescent signal at 520 nm (Arb units). Three replicates are depicted from each target. The three targets were 18s, FluA, and BH (Bordetella holmesii). Purified synthetic RNA molecules with sequences corresponding to BH, FluA, 18s, and R440 were used.

An assessment of sensitivity and specificity was made for the assay with regard to each of the three targets. This was done by selecting a population of spots from replicates either containing or lacking the intended target and comparing the individual spot emission to a threshold. For positive samples containing the target, 36 spots from the lowest tested concentration (6×10⁹ copies) were utilized. For negative samples, those lacking the target, 36 spots were selected from the multiplexed samples containing the alternate targets. The threshold was established as 3× the standard deviation of the local background emission plus the average local background emission. False negatives were registered as any spot emission lower than the threshold for a positive sample. False positives were registered as any spot emission higher than the threshold for a negative sample. The assay exceeded an 85% sensitivity/specificity criteria, for 18s and BH, but fell short for FluA with 11/36 false negatives. This shortfall in FluA was mainly a result of the low signal observed in the second replicate at 6×10⁹ copies. Sensitivity and specificity for FluA may improve if assessed at higher copy numbers. Table 3 summarizes the sensitivity/specificity results.

TABLE 3
Sensitivity and Specificity for the Assay

| | | Total | | | Above 85% |
|---|---|---|---|---|---|
| | Sample | Samples | FN | FP | Sensitivity/ |
| Target | Type | Spots | Spots | Spots | Specificity |
| 18s | Positive | 36 | 0 | N/A | Yes |
| 18s | Negative | 36 | N/A | 1 | Yes |
| FluA | Positive | 36 | 11 | N/A | No |
| FluA | Negative | 36 | N/A | 0 | Yes |
| BH | Positive | 36 | 0 | N/A | Yes |
| BH | Negative | 36 | N/A | 0 | Yes |

### Example 4. Multiplex Method of Detecting Target Nucleic Acids Using Quantum Dot Labels

A different version of the d-gRNA/Cas13 microarray assay was developed to increase signal amplification. This was done through the introduction of quantum dots as an enhanced optical emitter. Quantum dots (QD) were chosen due to their higher quantum efficiency over molecular fluorophores as well as their robustness to quenching. The stability of quantum dots under excitation allows them to be pumped such that, in principle, greater signal can be extracted by extending the excitation and detection times. To exploit quantum dots in the microarray assay a new target labeling motif was adopted. Target molecules were functionalized with biotin through a biotin pendant Mirus® reagent, as show in FIG. 8. The biotin labeled targets were then bound to immobilized d-gRNA/Cas13 complexes. A subsequent binding step anchored streptavidin functional quantum dots to the captured target molecules.

The choice of quantum dots selected for micro-array labeling was driven primarily by the maximum possible Stoke's shift. Accordingly, quantum dots that emit at 655 nm were chosen. These particles have a large absorbance between 300-400 nm allowing those skilled in optics to devise a filter set with a large gap of at least ˜255 nm.

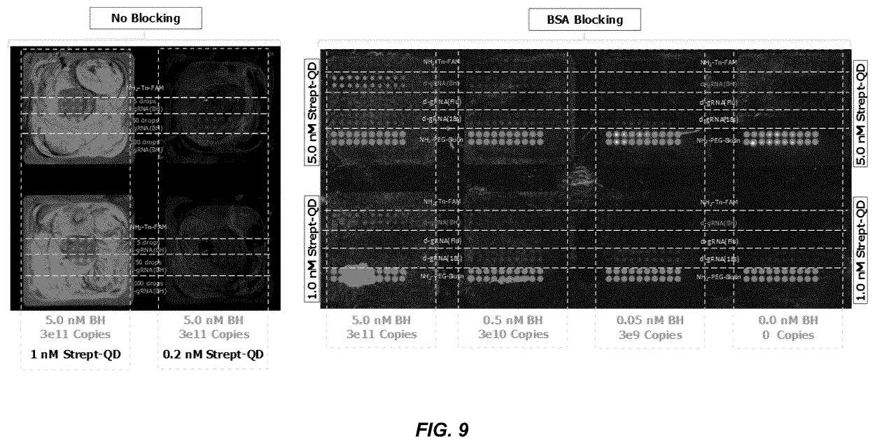

Initial attempts at labeling microarray captured targets with quantum dots demonstrated the need for proper blocking to prevent non-specific interactions between the quantum dots and the substrate surface. FIG. 9 shows, on the left, unblocked quantum dot labeling of d-gRNA (BH)/Cas subarrays after target capture for subarray with varying amounts of BH target. Without blocking, significant non-specific binding from the quantum dots was observed, as shown by high amorphous background seen across the microwell surface/bright blotchy emission across the microwell surface. Shown on the right, blocking with BSA was shown to reduce quantum dot non-specific binding and enable target specific signal from quantum dot labeling of on-target subarray for the BH-biotin target. Control subarrays of NH₂-PEG-Biotin were included to verify efficient binding of between streptavidin conjugated quantum dots and immobilized biotin functionality. As demonstrated in FIG. 9, implementing BSA blocking greatly reduced overall quantum dot non-specific binding and provided the first demonstration of quantum dot labeled, microarray captured synthetic target. This was realized in the observation of target specific emission from the on-target d-gRNA (BH) (a bacterial pathogen, Bordetella holmesii) sub-array upon binding synthetic BH-biotin to the microarray. Purified synthetic RNA molecules with sequences corresponding to BH were used in the assay. Signal was clearly observed when 5 nM quantum dots were used to label 3e11 copies (5 nM) of BH-biotin and faintly detected at 3c10 copies (0.5 nM). While apparent, the signal for bound target was lower than expected. One potential cause for reduced signal might have been degradation of the gRNAs and targets by nuclease contamination of the BSA blocker used during quantum dot binding.

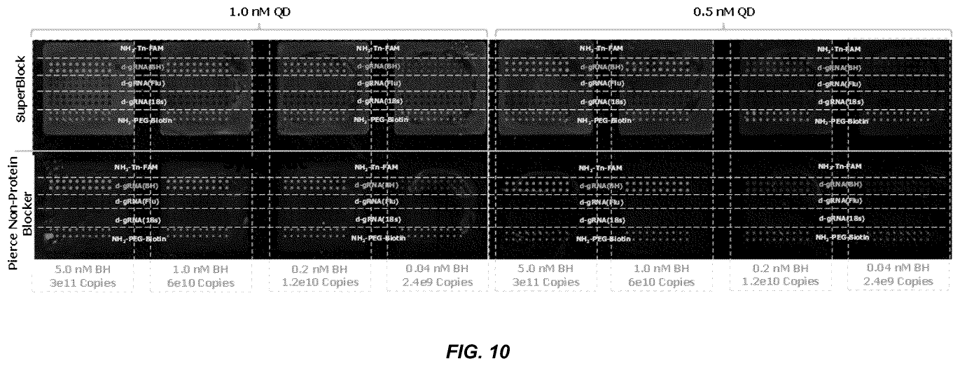

To avoid such degradation of the RNA components, different quantum dot blocking systems were evaluated as shown in FIG. 10. In FIG. 10, the image shows on-target emission for synthetic BH-biotin captured by d-gRNA (BH)/Cas subarrays. Here the quantum dot labeling step was carried out in SuperBlock™ or a non-protein blocker. Target was detected down to 0.04 nM/2.4c9 copies (vs 5 nM/3e11 copies with BSA). Lower concentrations of quantum dots also produced less background, but with reduced emission, especially at lower target concentration. Additionally, little cross-talk was observed to off-target subarrays. In each case, clear signal was observed for the on-target subarray (d-gRNA (BH)) over the entire range of target concentrations when labeled with 1.0 nM QD. The improvement in signal between FIG. 9 and FIG. 10 was attributed to 2 factors: the first being better preservation of d-gRNA by lack of nuclease activity during the quantum dot labeling step; the second being an improved background due to lower non-specific binding of the quantum dots to the substrate in the case of blocking of non-protein blocker. Accordingly, non-protein blocker was adopted as the blocker for the quantum dot labeling step.

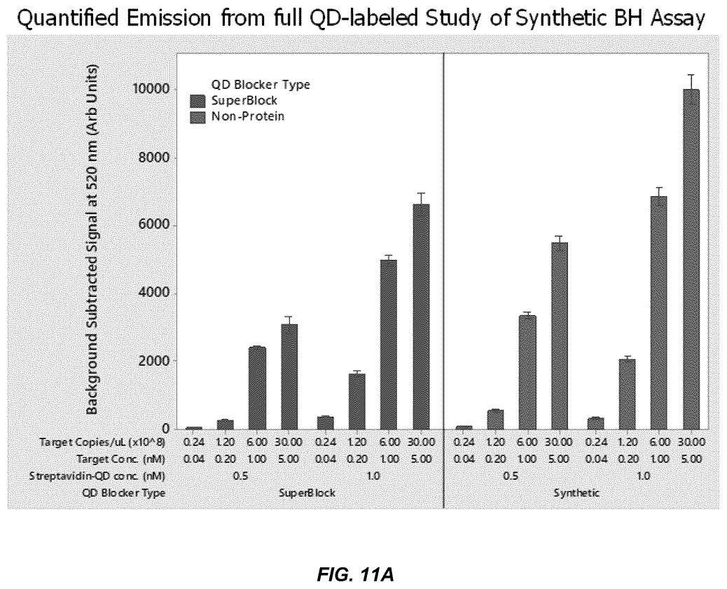

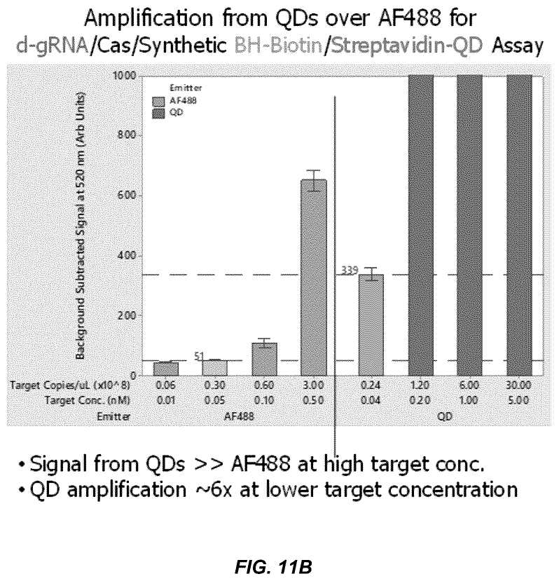

Signal from the assay in FIG. 10 is quantified in plots depicted in FIGS. 11A and 11B. This quantification in FIG. 11A shows a monotonic response to the synthetic BH-biotin target for both blocking conditions (SuperBlock™ blocker and for non-Protein blocker). Overall, higher signal was observed with increased QD conc. in non-protein blocker. The y-axis shows background subtracted signal at 520 nm (Arb units). Furthermore, the extent of signal amplification realized by labeling with quantum dots over Alexa Fluor® 488 is illustrated in FIG. 11B. For the lowest comparable concentrations (BH-AF488=0.05 nM, BH-Biotin=0.04 nM) there is an approximate 6× increase in signal for BH-Biotin target labeled with quantum dots compared to the directly labeled BH-AF488.

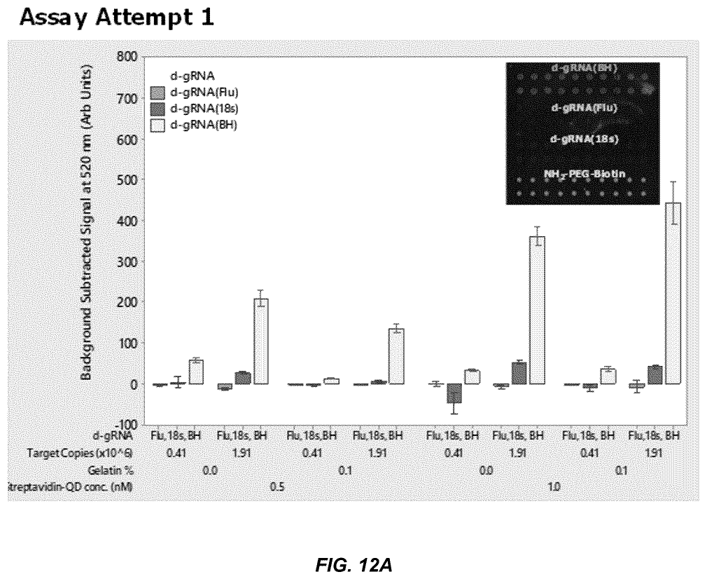

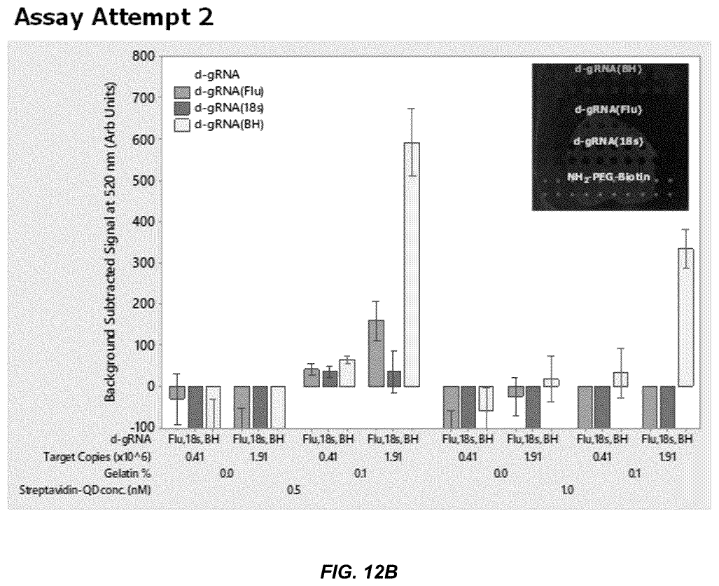

After refinement of the quantum dot binding conditions utilizing synthetic BH target, the labeling system was applied to genomic BH. For the genomic target assays, bovine gelatin was introduced to help disrupt non-specific interactions of labeled molecules with off-target d-gRNA sub arrays as well as the underlying substrate. Data from quantified results for two assays are displayed in FIGS. 12A and 12B, which shows quantification of signal for the detection of genomic BH (Bordetella holmesii)-biotin with quantum dot labeling. Plots for 2 attempts at the detection of genomic BH-biotin are depicted with inclusion of gelatin as a screening agent against non-specific target interactions. Each plot contains background subtracted emission for on-target d-gRNA (BH) subarrays as well as off-target (d-gRNA (Flu), d-gRNA (18s)) subarrays. In attempt 1, FIG. 12A, signal for genomic BH was realized under all conditions as distinct from off-target subarrays. However, some cross-talk to the d-gRNA (18s) was observed and gelatin showed a minor reduction to background from quantum dot non-specific binding. For attempt 2, FIG. 12B, non-specific binding of quantum dots was substantially higher with signal only cleanly observed for conditions involving 1.0 nM quantum dots and 0.1% gelatin. All other conditions were marked by interfering factors such as high background, high cross-talk or both. Signal was reduced without gelatin due to significant quantum dot background emission. With gelatin, distinct signal was observed at higher QD concentrations but obscured by cross-talk when less QDs were applied. Detection of biotin labeled genomic BH appeared successful for higher concentrations while detection of lower concentrations was inconsistent.

### Example 5. Multiplexing with and without Complexed dCas Enzyme

To evaluate the effect of capturing targets by direct hybridization with immobilized gRNA, a study was performed in which the Cas enzyme complexation step was omitted for certain wells on a gRNA functional surface. The general procedure was carried out as described in Example 1 with modifications mentioned in Example 4 for the implementation of quantum dots.

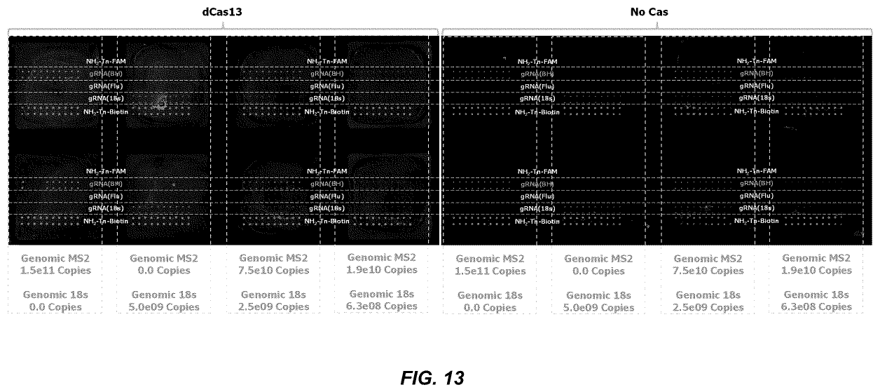

In this example, a portion of the slide (wells 1-8) was complexed with dCas13 enzyme as performed in the nominal assay procedure while, for the remaining portion of the slide (wells 9-16), no enzyme was added to the 1× MCLAB reaction buffer during the complexation step. Hereafter, target solutions containing genomic Bordetella holmesii or 18s in single-plex and duplex at different concentrations were incubated in the wells. Each well contained microarrays of gRNA (BH), gRNA (flu), and gRNA (18s). After quantum dot labeling and final washes, the slide was imaged at 635 nm excitation. FIG. 13 depicts an image of the assay showing signal for wells with dCas13 on the left and wells without dCas13 on the right. In all cases, signal is observed for each target in both single and multiplex regardless of the presence of Cas enzyme. Signal in the wells with lowest target concentration was very faint. The extent of background emission is notably higher in the wells that contain dCas13.

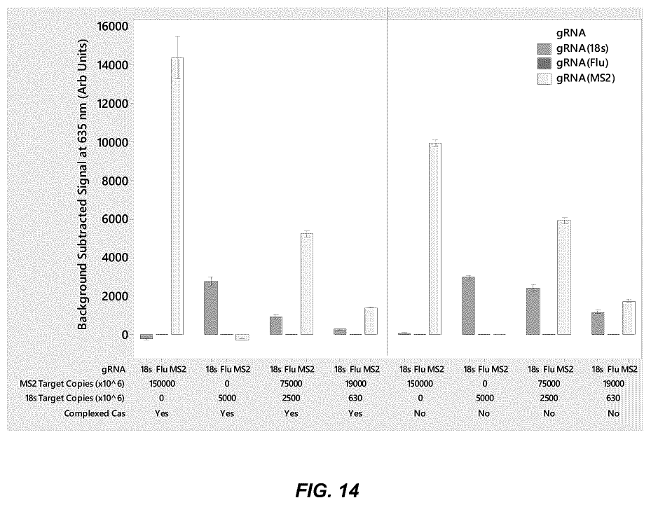

Quantified results for this study are depicted in FIG. 14. Except for the highest concentration singleplex MS2 wells, the signal for captured target was higher in the wells without Cas enzyme. This is especially pronounced for wells containing lower target concentrations of 18s for which the signal was 3×-4× higher without dCas13. The difference in signal is mainly attributed to the lower background noise in the wells without Cas enzyme.

### NON-LIMITING EMBODIMENTS OF THE DISCLOSURE

Embodiment 1 is a method of detecting a target nucleic acid in a sample, comprising: labeling a target nucleic acid with a label; after labeling the target nucleic acid, applying the labeled target nucleic acid to a microarray; on the microarray, incubating the target nucleic acid with: a guide RNA (gRNA) bound, directly or indirectly, to a solid support, wherein at least a portion of the gRNA is complementary to the target nucleic acid; and a deactivated Cas enzyme, wherein the gRNA and the Cas enzyme form a complex; washing the microarray; and detecting the target nucleic acid.

Embodiment 2 is a method of detecting multiple target nucleic acids in a sample, comprising: labeling multiple target nucleic acids with a label; after labeling the multiple target nucleic acids, applying the labeled target nucleic acids to a microarray comprising multiple identifiable locations; on the microarray, incubating the labeled target nucleic acids with: a first guide RNA (gRNA) bound, directly or indirectly, to a first identifiable location on a solid support, wherein at least a portion of the first gRNA is complementary to a first target nucleic acid; a second gRNA bound, directly or indirectly, to a second identifiable location on the solid support, wherein at least a portion of the second gRNA is complementary to a second target nucleic acid; optionally one or more additional gRNA bound to one or more additional identifiable locations, wherein at least a portion of the one or more additional gRNA is complementary to one or more additional target nucleic acids, respectively, and deactivated Cas enzymes, wherein each first gRNA forms a complex with a deactivated Cas enzyme and wherein each second gRNA forms a complex with a deactivated Cas enzyme; washing the microarray; and detecting the nucleic acids by detecting the presence or absence of a signal from the identifiable locations on the microarray.

Embodiment 3 is a method of embodiment 1 or 2, wherein the label is attached to the target nucleic acid using a linker such as Mirus® linker, EZ-Link™ Sulfo-NHS-Biotin, Thermo Scientific™ EZ-Link™ PFP-Biotin, PHOTOPROBE® Biotin for Nucleic Acid Labeling, 3′ EndTag™ DNA End Labeling System, FlashTag™ Biotin HSR RNA Labeling Kit, and/or Ulysis™ Nucleic Acid Labeling Kit.

Embodiment 4 is the method of any one of embodiments 1-3, wherein the label is covalently attached to the target nucleic acid.

Embodiment 5 is the method of any one of embodiments 1-4, wherein the label is attached to the target nucleic acid using a linker, wherein the linker incorporates a quaternary amine.

Embodiment 6 is the method of any one of embodiments 1-5, wherein the label is attached to the target nucleic acid using a Mirus® linker.

Embodiment 7 is a method of detecting a target nucleic acid in a sample, comprising: applying the target nucleic acid to a microarray; on the microarray, incubating the target nucleic acid with: a guide RNA (gRNA) bound, directly or indirectly, to a solid support, wherein at least a portion of the gRNA is complementary to the target nucleic acid; and a deactivated Cas enzyme, wherein the gRNA and the Cas enzyme form a complex; applying a labeled complementary nucleic acid, wherein the labeled complementary nucleic acid is complementary to a portion of the target nucleic acid; washing the microarray; and detecting the target nucleic acid.

Embodiment 8 is a method of detecting multiple target nucleic acids in a sample, comprising: applying the target nucleic acids to a microarray comprising multiple identifiable locations; on the microarray, incubating the target nucleic acids with: a first guide RNA (gRNA) bound, directly or indirectly, to a first identifiable location on a solid support, wherein at least a portion of the first gRNA is complementary to a first target nucleic acid; a second gRNA bound, directly or indirectly, to a second identifiable location on the solid support, wherein at least a portion of the second gRNA is complementary to a second target nucleic acid; optionally one or more additional gRNA bound to one or more additional identifiable locations, wherein at least a portion of the one or more additional gRNA is complementary to one or more additional target nucleic acids, respectively, and deactivated Cas enzymes, wherein each first gRNA forms a complex with a deactivated Cas enzyme and wherein each second gRNA forms a complex with a deactivated Cas enzyme; applying a first, second, and one or more additional labeled complementary nucleic acids, wherein the first labeled complementary nucleic acid is complementary to a portion of the first target nucleic acid, wherein the second labeled complementary nucleic acid is complementary to a portion of the second target nucleic acid, and wherein the one or more additional labeled complementary nucleic acids are complementary to a portion of the one or more additional target nucleic acids, respectively; washing the microarray; and detecting the nucleic acids by detecting the presence or absence of a signal from the identifiable locations on the microarray.

Embodiment 9 is a microarray for identifying the presence of a target nucleic acid comprising: at least one identifiable location on a solid support; a guide RNA (gRNA) bound to an identifiable location on a solid support via amine/epoxy coupling, wherein the gRNA is 5′ amine-modified, further wherein at least a portion of the gRNA is complementary to the target nucleic acid; a deactivated Cas enzyme; wherein the gRNA and the deactivated Cas enzyme form a complex.

Embodiment 10 is the method or microarray of any one of embodiments 1-9, wherein the deactivated Cas enzyme is a deactivated Cas13 enzyme.

Embodiment 11 is the method or microarray of any one of embodiments 1-9, wherein the deactivated Cas enzyme is a deactivated Cas12 enzyme.

Embodiment 12 is the method or microarray of any one of embodiments 1-9, wherein the deactivated Cas enzyme is a deactivated Cas9 enzyme.

Embodiment 13 is the method or microarray of any one of embodiments 1-12, wherein the deactivated Cas enzyme is deactivated using deactivating guide RNA.

Embodiment 14 is the method or microarray of any one of embodiments 1-12, wherein the deactivated Cas enzyme comprises one or more mutations that silence nucleic acid cleavage activity.

Embodiment 15 is the method or microarray of any one of embodiments 1-14, wherein the microarray comprises a solid support comprising glass, a semiconductor (e.g., silicon, silicon nitride), metal (e.g., gold, platinum), carbon (graphite, graphene), a polymer (e.g., polycarbonate), cellulose, and/or a metal oxide (e.g., ITO, TiO2).

Embodiment 16 is the method or microarray of embodiment 15, wherein the solid support comprises glass.

Embodiment 17 is the method or microarray of any one of embodiments 1-16, wherein the target nucleic acid is RNA.

Embodiment 18 is the method or microarray of any one of embodiments 1-16, wherein the target nucleic acid is DNA.

Embodiment 19 is a kit comprising:

a microarray comprising a guide RNA (gRNA) bound to a solid support via amine/epoxy coupling, wherein the gRNA is 5′ amine-modified, further wherein at least a portion of the gRNA is complementary to a target nucleic acid; a deactivated Cas enzyme; a nucleic acid label or a labeled complementary nucleic acid, wherein the labeled complementary nucleic acid is complementary to a portion of the target nucleic acid; and blocking solution.

Embodiment 20 is kit comprising: a microarray comprising: a first guide RNA (gRNA) bound to a solid support via amine/epoxy coupling to a first identifiable location on the solid support, wherein the first gRNA is 5′ amine-modified, further wherein at least a portion of the first gRNA is complementary to a first target nucleic acid; a second guide RNA (gRNA) bound to the solid support via amine/epoxy coupling to a second identifiable location on the solid support, wherein the first gRNA is 5′ amine-modified, further wherein at least a portion of the second gRNA is complementary to a second target nucleic acid; optionally one more additional guide RNA (gRNA) bound to the solid support via amine/epoxy coupling to one or more additional identifiable locations on the solid support, wherein the one or more additional gRNAs are 5′ amine-modified, further wherein at least a portion of the one or more additional gRNAs are complementary to one or more additional target nucleic acids, respectively; a deactivated Cas enzyme; a nucleic acid labels or a first second, and one or more additional labeled complementary nucleic acids, wherein the first labeled complementary nucleic acid is complementary to a portion of the first target nucleic acid, wherein the second labeled complementary nucleic acid is complementary to a portion of the second target nucleic acid, and wherein the one or more additional labeled complementary nucleic acids are complementary to a portion of the one or more additional target nucleic acids, respectively; and blocking solution.

Embodiment 21 is the kit of embodiment 19 or 20, wherein the blocking solution is free of, or nearly free of, RNase activity, or wherein the RNase activity has been neutralized or reduced.

Embodiment 22 is the kit of any one of embodiments 19-21, wherein the blocking solution comprises SuperBlock™ blocking buffer.

Embodiment 23 is the kit of any one of embodiments 19-22, wherein the nucleic acid label comprises a fluorescent molecule.

Embodiment 24 is the kit of any one of embodiments 19-22, wherein the nucleic acid label comprises a quantum dot.

Embodiment 25 is the kit of embodiment 24, wherein the nucleic acid label comprises Alexa Fluor® 488.

Embodiment 26 is the kit of any one of embodiments 19-22, wherein the nucleic acid label comprises biotin.

Embodiment 27 is the kit of any one of embodiments 19-26, wherein the kit further comprises Mirus® reagent for attaching the nucleic acid label to the target nucleic acid.

Embodiment 28 is the kit of any one of embodiments 19-26, wherein the kit further comprises a Thermo Scientific™ EZ-Link™ Sulfo-NHS-Biotin kit, a Thermo Scientific™ EZ-Link™ PFP-Biotin kit, a PHOTOPROBE® Biotin for Nucleic Acid Labeling kit, a Vector Laboratories 3′ EndTag™ DNA End Labeling System, an Applied Biosystems™ FlashTag™ Biotin HSR RNA labeling kit, or an Invitrogen Ulysis™ nucleic acid labeling kit for attaching the nucleic acid labels to the target nucleic acid.

The teachings of all patents, published applications and references cited herein are incorporated by reference in their entirety.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

While example embodiments have been particularly shown and described, it will be understood by those skilled in the art that various changes in form and details may be made therein without departing from the scope of the embodiments.

1. A method of detecting a target nucleic acid in a sample, comprising:
providing a sample comprising a target nucleic acid;
contacting the sample with an apparatus comprising a plurality of guide RNA (gRNA) molecules bound, directly or indirectly, to a solid support, wherein at least one gRNA molecule in the plurality comprises a nucleotide sequence that is complementary to a portion of the target nucleic acid in the sample, and wherein the contacting is under conditions in which the target nucleic acid hybridizes to the complementary gRNA molecule on the apparatus;
washing the apparatus to remove unhybridized nucleic acids; and
detecting the target nucleic acid.

2. The method of claim 1, wherein the sample is a biological sample.

3. The method of claim 2, wherein the biological sample comprises a pathogen or nucleic acid from a pathogen.

4. The method of claim 2, wherein the biological sample is a clinical sample from a subject or group of subjects.

5. The method of claim 1, wherein the target nucleic acid is DNA.

6. The method of claim 1, wherein the target nucleic acid is RNA.

7. (canceled)

8. The method of claim 1, wherein the target nucleic acid comprises a label, wherein the label comprises a quantum dot, a fluorescent molecule, or biotin.

9.-13. (canceled)

14. The method of claim 1, wherein the target nucleic acid is present in the sample at a concentration of about 1 nM or less.

15.-17. (canceled)

18. The method of claim 1, wherein the apparatus is a microarray comprising multiple identifiable locations.

19. (canceled)

20. The method of claim 1, wherein the plurality of gRNA molecules on the apparatus are modified.

21. (canceled)

22. The method of claim 1, wherein the plurality of gRNA molecules on the apparatus are in a complex with a CRISPR-associated (Cas) enzyme.

23. The method of claim 22, wherein the Cas enzyme is a deactivated Cas enzyme.

24. (canceled)

25. The method of claim 23, wherein the deactivated Cas enzyme is a deactivated Cas9 enzyme, a deactivated Cas12 enzyme, or a deactivated Cas13 enzyme.

26. (canceled)

27. The method of claim 1, wherein the apparatus is incubated with a blocking solution prior to detecting the target nucleic acid.

28.-31. (canceled)

32. A microarray comprising a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid.

33. The microarray of claim 32, wherein the solid support comprises glass, a semiconductor, a metal, carbon, a polymer, cellulose, a metal oxide, or a combination thereof.

34. (canceled)

35. (canceled)

36. The microarray of claim 32, wherein the plurality of gRNA molecules on the apparatus are in a complex with a CRISPR-associated (Cas) enzyme.

37. The microarray of claim 36, wherein the Cas enzyme is a deactivated Cas enzyme.

38. (canceled)

39. (canceled)

40. A kit comprising:
a microarray comprising a plurality of guide RNA (gRNA) molecules bound, directly or indirectly, to a solid support, wherein the microarray comprises multiple identifiable locations, wherein each identifiable location comprises a gRNA molecule having a nucleotide sequence that is complementary to the nucleotide sequence of at least one target nucleic acid.

41.-48. (canceled)

49. The method of claim 1,
wherein the target nucleic acid comprises a label and is present in the sample at a concentration of about 1 nM or less; and
the sample is contacted with a microarray comprising a plurality of guide RNA (gRNA) molecules covalently bound to a solid support, for a time period of about 24 hours or less, wherein:
the plurality of gRNA molecules are in a complex with a deactivated CRISPR-associated (Cas) enzyme, and
at least one gRNA molecule in the plurality comprises a nucleotide sequence that is complementary to a portion of the target nucleic acid in the sample.

50.-53. (canceled)
